(19) United States
(12) Patent Application Publication
(10) Pub. No.: US 2024/0181260 A1
(43) Pub. Date: Jun. 6, 2024

(54) IMPLANTABLE MEDICAL DEVICE (IMD) INCLUDING SENSING AMPLIFIER CIRCUITRY

(71) Applicant: Advanced Neuromodulation Systems, Inc., Plano, TX (US)

(72) Inventor: Michael Fortner, Plano, TX (US)

(21) Appl. No.: 18/491,267

(22) Filed: Oct. 20, 2023

Related U.S. Application Data
(60) Provisional application No. 63/417,839, filed on Oct. 20, 2022.
(60) Provisional application No. 63/429,916, filed on Dec. 2, 2022.

(51) Int. Cl. A61N 1/36; A61N 1/02; A61N 1/05; A61N 1/06

(52) U.S. Cl. CPC A61N 1/36139; A61N 1/025; A61N 1/0551; A61N 1/06; A61N 1/36062; A61N 1/36071; A61N 1/36171; A61N 1/36175; A61N 1/36178

(57) ABSTRACT

An implantable medical device (IMD) configured to sense biosignals of a patient. The IMD comprises one or more power components for powering the IPG and sensing circuitry for sensing one or more biosignals of the patient, wherein the sensing circuitry comprises a hybrid circuit having a BJT portion and a CMOS FET portion, the BJT portion configured to amplify low voltage signals with low equivalent noise and the CMOS FET portion forming a power-optimized output stage.

### RELATED APPLICATIONS

This application claims the benefit of U.S. Provisional Application No. 63/417,839, filed Oct. 20, 2022, entitled “AN IMPLANTABLE MEDICAL DEVICE (IMD) COMPRISING SENSING CIRCUITRY WITH AMPLIFIER FOR AMPLIFYING LOW VOLTAGE SIGNALS AND METHODS OF OPERATION” and U.S. Provisional Application No. 63/429,916, filed Dec. 2, 2022, entitled “IMPLANTABLE MEDICAL DEVICE (IMD) INCLUDING SENSING AMPLIFIER CIRCUITRY” which are incorporated herein by reference.

### TECHNICAL FIELD

The present application generally relates to sensing one or more biosignals using sensing circuitry of an implantable medical device (IMD) such as an implantable pulse generator (IPG) for stimulating neural tissue of a patient.

### BACKGROUND

Implantable medical devices are used for a wide variety of medical conditions. For example, a number of implantable medical devices have been commercially distributed that allow electrical pulses or signals to be controllably delivered to targeted tissue or nerves after implantation of the respective device within a patient. Such implantable medical devices may be used for cardiac pace making, cardiac rhythm management, treatments for congestive heart failure, implanted defibrillators, and neurostimulation. Neurostimulation encompasses a wide range of applications, such as for example, treatment of chronic pain, treatment of motor disorders, treatment of incontinence and other sacral nerve related disorders, reduction of epileptic seizures, and treatment of depression.

Neurostimulation in the form of spinal cord stimulation (SCS), for example, has been used as a treatment for chronic pain for a number of years. SCS is often used to alleviate pain after failed surgery, pain due to neuropathies, or pain due to inadequate blood flow. In accordance with SCS therapy, non-nociceptive fibers are stimulated to alleviate pain symptoms in cases of chronic pain.

Implantable electrical stimulation devices generally include an implanted pulse generator that generates electrical pulses or signals that are transmitted to targeted tissue or nerves through a therapy delivery element, such as a lead with an electrode array. In the case of SCS, an electrode array present on a distal end of a lead may be implanted so as to be disposed within the epidural space for delivery of the electrical stimulation. A pulse generator coupled to a proximal end of the lead may thus be enabled to apply neural stimuli to the dorsal column in order to give rise to a compound action potential (CAP). The dorsal column contains the afferent A-beta (Aβ) fibers to mediate sensations of touch, vibration, and pressure from the skin, whereby ones of the Aβ fibers may be therapeutically recruited by the neural stimuli provided through the electrode array by the pulse generator.

According to conventional SCS, stimulation pulses are provided to neural tissue of the dorsal column in a regular pattern with each pulse having a predetermined amplitude (e.g., current intensity) and being separated by a fixed inter-pulse interval that defines a stimulation frequency configured for inducing a tingling sensation (known medically as paresthesia) in the patient. For example, stimulation of the Aβ fibers may induce paresthesia and therefore may provide the mechanism of action for traditional tonic SCS to mask the pain. Although the paresthesia can be uncomfortable or even painful in patients, the paresthesia is often substantially more tolerable than the pain otherwise experienced by the patients.

A more recent approach to pain management through SCS is to use high-frequency SCS (HFSCS) to provide paresthesia-free therapy. HFSCS typically includes pulses at frequencies between 1500 Hz and 10,000 Hz although even higher frequencies could be used. In accordance with HFSCS, high-frequency electrical pulses are delivered at a current intensity below the paresthesia threshold. For example, HFSCS stimulation regimens implementing a stimulation frequency of up to 10 kHz have been found to be effective in providing pain relief without eliciting paresthesia (see e.g., Arie J E, Mei L, Carlson K W, and Shils J L, “High frequency stimulation of dorsal column axons: potential underlying mechanism of paresthesia-free neuropathic pain”, Poster at International Neuromodulation Society Conference, 2015; and Adnan Al-Kaisy, MD, Jean-Pierre Van Buyten, MD, Iris Smet, MD, Stefano Palmisani, MD, David Pang, MD, and Thomas Smith, MD, “Sustained Effectiveness of 10 kHz High-Frequency Spinal Cord Stimulation for Patients with Chronic, Low Back Pain: 24-Month Results of a Prospective Multicenter Study”, Pain Medicine, 2014, 15: 347-354; the disclosures of which are incorporated herein by reference.

Another approach to pain management through SCS uses a stimulation technique called burst stimulation. In implementation of burst stimulation therapy, packets (e.g., “bursts”) of high-frequency impulses are delivered periodically (e.g., five pulses at 500 Hz, delivered 40 times per second) at a current intensity below the paresthesia threshold. It has been found that such burst stimulation suppresses neuropathic pain at least as well as, and possibly better than, traditional tonic SCS stimulation and provides such pain relief without eliciting paresthesia. Burst stimulation that bypasses the paresthesia process is hypothesized to have a different mechanism of action than that of traditional tonic SCS stimulation, and therefore may bypass Aβ fiber activation (see e.g., Arie et al., “High frequency stimulation of dorsal column axons: potential underlying mechanism of paresthesia-free neuropathic pain”, incorporated by reference above; Beurrier, et al., “Subthalamic nucleus neurons switch from single-spike activity to burst-firing mode,” J. Neurosci., 19(2): 599-609, 1999; and Stefan Schu, MD, PhD, Philipp J. Slotty, MD, Gregor Bara, MD, Monika von Knop, Deborah Edgar, PhDt, Jan Vesper, MD, PhD, “A Prospective, Randomised, Double-blind, Placebo-controlled Study to Examine the Effectiveness of Burst Spinal Cord Stimulation Patterns for the Treatment of Failed Back Surgery Syndrome”, Neuromodulation 2014; 17: 443-450; the disclosures of which are incorporated herein by reference.

Irrespective of the particular SCS stimulation technique implemented, stimuli amplitude (e.g., current intensity) and/or delivered charge are conventionally maintained below a comfort threshold, above which recruitment of Δβ fibers may be at a level so large as to produce discomfort and even pain in the patient, in order to provide comfortable operation for a patient. Correspondingly, stimuli amplitude and/or delivered charge are generally maintained above a recruitment threshold to recruit desired action potentials for providing effective therapy to the patient (e.g., inducing an analgesic effect whereby the patient experiences no pain, or a relatively small amount of pain, at the region of interest). Additionally, in accordance with burst stimulation techniques, stimuli amplitude and/or delivered charge are maintained below a paresthesia threshold.

Maintaining neural recruitment at an appropriate level for effectiveness of SCS and related neurostimulation therapies can be challenging due to various events, such as electrode migration and/or postural changes of the patient, that can alter the neural recruitment with respect to a particular stimulus. For example, there is room in the epidural space for an electrode array to move, whereby such movement of the electrodes may alter a distance between the electrode and one or more fibers resulting in changes to the recruitment efficacy of a particular stimulus. Additionally, the spinal cord itself may move within the cerebrospinal fluid (CSF) with respect to the dura, such as due to postural changes of the patient, whereby the distance and/or the amount of CSF between the spinal cord and the electrodes may change resulting in changes to the recruitment efficacy of a particular stimulus.

Measurement of evoked compound action potentials (ECAPs) provides a means of directly assessing the level of fiber recruitment in the dorsal columns of the spinal cord. ECAPs are signals elicited by electrical stimulations and recorded near a bundle of fibers. In particular, ECAPs usually arrive less than one millisecond (<1 ms) after a corresponding stimulation pulse and last in the range of approximately one half to one millisecond (0.5-1.0 ms). ECAPs may be measured and analyzed, for example, to evaluate and/or control the comfort and efficacy of a SCS treatment regimen (see e.g., US patent publication numbers 2020/0282208 A1 entitled “Neural Stimulation Dosing”; 2011/0184488 A1 entitled “Spinal Cord Stimulation to Treat Pain”; and 2020/0391031 A1 entitled “System and Method to Managing Stimulation of Select A-Beta Fiber Components”; the disclosures of which are incorporated herein by reference).

### SUMMARY

Embodiments of the present patent disclosure are directed to systems, circuitry, and associated methods for facilitating neurostimulation and biosignal sensing. Some example implementations may involve monitoring or sensing of response signals generated by the target tissue of a patient in response to stimulation. Some example implementations may involve sensing of biosignals using a compact, ultra-low-noise, ultra-low-power, nerve action amplifier realized through hybridization of different semiconductor technologies, e.g., bipolar and CMOS technologies. In an example configuration, by utilizing of low-noise-optimized bipolar transistors in conjunction with power-optimized integrated CMOS circuitry, a hybrid technology sensing amplifier assembly may be configured to provide a performance level that may advantageously exceed the performance levels of either technology when used separately in an amplifier design.

In one aspect, an IPG for generating electrical pulses to stimulate a neural tissue of a patient is disclosed. The IPG may comprise, inter alia, one or more battery components for powering the IPG; one or more stimulation engines or sets of pulse generating circuitry for generating stimulation pulses; a header structure with a plurality of electrical connections adapted to contact one or more terminals of one or more stimulation leads or one or more lead extensions coupled to respective ones of the one or more stimulation leads, wherein the one or more stimulation leads each include a plurality of electrodes; and sensing circuitry for sensing one or more biosignals associated with the neural tissue, wherein the sensing circuitry comprises an amplifier for amplifying the one or more biosignals and the amplifier includes a noise-optimized input stage coupled to a power-optimized output stage. In one example arrangement, the noise-optimized input stage may comprise one or more bipolar junction transistors (BJTs) formed on a first semiconductor die and the power-optimized output stage may comprise one or more complementary metal-oxide-semiconductor (CMOS) field effect transistors (FETs) formed on a second semiconductor die. In one example arrangement, the one or more BJTs comprise BJTs having a forward transfer function greater than a predetermined value, e.g., a forward current gain (P) greater than around 400.

In another aspect, an IMD for sensing biosignals of a patient is disclosed. The IMD may comprise, inter alia, a power supply for powering the IMD; at least one lead having a plurality of electrodes; and sensing circuitry including an amplifier comprising a first circuit portion formed of one or more BJTs disposed on a first semiconductor die and a second circuit portion electrically coupled to the first circuit portion, the second circuit portion formed of a plurality of CMOS FETs disposed on a second semiconductor die, wherein the first circuit portion is configured to receive a biosignal from the patient via one or more electrodes switchably selected from the plurality of electrodes. In one arrangement, the biosignal may comprise at least one evoked compound action potential (ECAP) signal generated by a nerve tissue of the patient in response to receiving one or more stimulation pulses. In another arrangement, the biosignal may comprise at least local field potential signal associated with the nerve tissue.

In another aspect, an example biostimulation system is disclosed, wherein the system is operable with a patient controller (PC) device, a clinician programmer (CP) device, and/or any suitable external device, the system configured to sense various types of biosignals using a hybrid sensing amplifier (SA) as described herein.

In still further aspects, one or more embodiments of a non-transitory computer-readable medium, computer program product or distributed storage media containing computer-executable program instructions or code portions stored thereon are disclosed for effectuating one or more embodiments herein when executed by a processor entity of a patient controller device, a clinician programmer device, or an IPG/IMD, and the like, mutatis mutandis.

Additional features, benefits and advantages of the embodiments will be apparent in view of the following description and accompanying Figures.

### BRIEF DESCRIPTION OF THE DRAWINGS

Embodiments of the present disclosure are illustrated by way of example, and not by way of limitation, in the Figures of the accompanying drawings in which like references indicate similar elements. It should be noted that different references to “an” or “one” embodiment in this disclosure are not necessarily to the same embodiment, and such references may mean at least one. Further, when a particular feature, structure, or characteristic is described in connection with an embodiment, it is submitted that it is within the knowledge of one skilled in the art to effectuate such feature, structure, or characteristic in connection with other embodiments whether or not explicitly described.

The accompanying drawings are incorporated into and form a part of the specification to illustrate one or more exemplary embodiments of the present disclosure. Various advantages and features of the disclosure will be understood from the following Detailed Description taken in connection with the appended claims and with reference to the attached drawing Figures in which:

FIG. 1A shows an example stimulation system that may utilize embodiments of sensing signal stimulation according to some examples of the present invention;

FIGS. 1B and 1C show an environment in which stimulation systems implementing sensing signal stimulation of embodiments of the present invention may be deployed;

FIG. 2 shows a functional block diagram of an example implantable pulse generator adapted to elicit sensing signals according to embodiments of the present invention;

FIG. 3 shows a graph representing a burst stimulation waveform of a paresthesia-free stimulation technique;

FIG. 4 shows a flow diagram of operation according to an example process to elicit sensing signals in association with corresponding therapeutic neural stimuli according to embodiments of the present invention;

FIGS. 5-9 show graphs representing interleaved implementations of pinging-pulses delivered in association with therapeutic neural stimuli according to embodiments of the present invention;

FIGS. 10-12 show graphs representing postfixed implementations of pinging-pulses delivered in association with therapeutic neural stimuli according to embodiments of the present invention;

FIG. 13 shows a flow diagram of operation according to an example process in which sensing signals elicited by pinging-pulses are utilized in an open-loop implementation for configuration of a therapeutic stimulation regimen according to embodiments of the present invention;

FIG. 14 shows a flow diagram of operation according to an example process in which sensing signals elicited by pinging-pulses are utilized in a closed-loop implementation for configuration of a therapeutic stimulation regimen according to embodiments of the present invention;

FIG. 15 depicts a graph representing interleaved implementations of pinging-pulses delivered in association with therapeutic neural stimuli according to embodiments of the present invention;

FIG. 16 depicts a flowchart for processing sensor data to isolate ECAP features for a neurostimulation therapy according to some embodiments;

FIG. 17 depicts a waveform representing sensed electrical activity corresponding to an electrical pulse and its evoked compound action potential for processing according to some embodiments;

FIG. 18 depicts respective graphs of sensor data in the frequency-temporal domain and associated feature identification according to some embodiments;

FIGS. 19 and 20 depict isolated ECAP features with surrounding areas masked with Gaussian noise according to some embodiments;

FIG. 21A depicts a graph representing raw signal data obtained by sensing circuitry of an IPG and a graph representing a reconstructed signal that may include segmented ECAP features according to some embodiments;

FIG. 21B depicts a scheme for additional processing of raw data for obtaining a reconstructed signal according to some embodiments;

FIG. 22 depicts a patient system computational model for effectuating recovery of ECAP features from a sensing electrode according to some embodiments;

FIG. 23 depicts operations for conducting ECAP sensing operations for a neurostimulation system according to some representative embodiments;

FIG. 24 depicts an ECAP display according to some embodiments;

FIG. 25 depicts a clinician user interface with display of ECAP data to assist an implant procedure according to some embodiments;

FIG. 26 depicts the propagation of ECAP from stimulation electrodes occurring rostrally along respective recording electrodes along the depicted stimulation lead for processing according to some embodiments;

FIG. 27 depicts a graph showing a phase shift or latency change for processing according to some embodiments;

FIG. 28 depicts a graph showing the emergence and/or disappearance of a secondary phase of the ECAPs according to some embodiments and FIG. 29 depicts a graph showing the disappearance of a phase (P1) of ECAP signals according to some embodiments;

FIG. 30 depicts lead arrangements where the ECAP is generated by the activation of the dorsal column axons and the axonal activation is maximized when the electric field is aligned in the axonal direction according to some embodiments;

FIG. 31 depicts a block diagram of an IMD having one or more stimulation engines of sets of stimulation circuitry and associated sensing amplifier circuitry for sensing biosignals according to an example embodiment;

FIG. 32 depicts a block diagram of a pulse generator portion having pulse set control and lead electrode selection control wherein a sensing amplifier circuit may be switchably coupled to a portion of electrodes for sensing a biosignal from a tissue interface according to some embodiments;

FIG. 33 depicts a schematic diagram of a low noise amplifier circuit that may be provided as part of an implantable medical device for sensing biosignals according to an example embodiment;

FIG. 34 depicts a schematic diagram of a low noise amplifier circuit with additional details according to example embodiment; and

FIG. 35 depicts an example biostimulation system with respect to a two-electrode arrangement and associated switching components for coupling to an amplifier circuit of the present disclosure according to an embodiment.

### DETAILED DESCRIPTION

In the description herein for embodiments of the present disclosure, numerous specific details are provided, such as examples of circuits, devices, components and/or methods, to provide a thorough understanding of embodiments of the present disclosure. One skilled in the relevant art will recognize, however, that an embodiment of the disclosure can be practiced without one or more of the specific details, or with other apparatuses, systems, assemblies, methods, components, materials, parts, and/or the like set forth in reference to other embodiments herein. In other instances, well-known structures, materials, or operations are not specifically shown or described in detail to avoid obscuring aspects of embodiments of the present disclosure. Accordingly, it will be appreciated by one skilled in the art that the embodiments of the present disclosure may be practiced without such specific components. It should be further recognized that those of ordinary skill in the art, with the aid of the Detailed Description set forth herein and taking reference to the accompanying drawings, will be able to make and use one or more embodiments without undue experimentation.

Additionally, terms such as “coupled” and “connected,” along with their derivatives, may be used in the following description, claims, or both. It should be understood that these terms are not necessarily intended as synonyms for each other. “Coupled” may be used to indicate that two or more elements, which may or may not be in direct physical or electrical contact with each other, co-operate or interact with each other. “Connected” may be used to indicate the establishment of communication, i.e., a communicative relationship, between two or more elements that are coupled with each other. Further, in one or more example embodiments set forth herein, generally speaking, an electrical element, component or module may be configured to perform a function if the element may be programmed for performing or otherwise structurally arranged to perform that function.

Without limitation, examples relating to biosignal monitoring using a hybrid technology SA arrangement will be set forth below in the context of ECAP signal generation, sensing and processing, inter alia, in an SCS therapy system.

ECAP measurements, provided by a suitable sensing apparatus may afford a direct assessment of the neural recruitment and its variation over time. In some arrangements, ECAP measurements may be used to understand the relationship between the stimulation which is being delivered and the recruitment of the Δβ fibers of the spinal cord. There are many factors that may influence this relationship including, e.g., the stimulation frequency, amplitude, and pulse width, the presence of pharmacological agents, and the proximity of the electrode(s) to the cord, etc. Further, the patient's posture, heartbeat, breathing, and/or other activities may change the electrode positions, thereby altering the signal activity. Although it is known that reliable ECAP measurements are important in characterizing and/or modulating a therapy, extant signal sensing techniques are generally inadequate because of the presence of electrically noisy tissue environments coupled with low amplitudes of the generated signals in a biological model.

Sensing signal stimulation techniques are provided according to embodiments of the invention for use in sensing responsive signals with respect to the application of paresthesia-free stimulation in some implementations. For example, sensing signal initiators may be utilized with respect to implantable medical devices operable to controllably deliver electrical pulses or signals to targeted tissue or nerves after implantation of the respective device within a patient.

In the case of spinal cord stimulation (SCS), fibers that generate ECAP signals are generally the A-beta (Δβ) fibers located in the dorsal column. Accordingly, conventional measurement of ECAPs may be practical with respect to traditional tonic SCS, where stimulation of the Δβ fibers is performed at levels sufficient to induce paresthesia. For example, conventional ECAP sensing is known to measure the direct stimulation response to conventional tonic SCS to maintain a substantially constant level of paresthesia. However, the ECAPs for burst stimulation and high frequency stimulation may occur at sufficiently low levels that the ECAPs are not sufficient to provide an accurate assessment of the concurrent neural response. For example, burst stimulation may be provided at sufficiently low amplitudes to ensure that the patient does not experience paresthesia and thereby the resulting ECAPs do not generate an electrical field of sufficient strength for sensing using one or more electrodes of the stimulation lead. In these situations, ECAPs may not be present or may be of such low signal strength and/or present in a very low signal to noise ratio (SNR) so as to make their measurement and/or analysis impractical or even impossible.

To aid in understanding the concepts herein, a description of examples relating to implantable medical devices of a spinal cord stimulation (SCS) system is set forth below. However, it is to be understood that, while sensing signal stimulation techniques in accordance with concepts herein are well suited for applications in SCS, the disclosure in its broadest aspects is not so limited. Rather, sensing signal stimulation techniques of the disclosure may be used with various types of electronic stimulus delivery systems.

Sensing signal stimulation according to concepts herein may be utilized with one or more therapy delivery elements comprising an electrical lead including one or more electrodes to deliver pulses or signals to a respective target tissue site in a patient and one or more sensing electrodes to sense electrical signals at the target tissue site within the patient. In the various embodiments contemplated by this disclosure, therapy may include stimulation therapy, sensing or monitoring of one or more physiological parameters, and/or the like. A target tissue site may refer generally to the target site for implantation of a therapy delivery element, regardless of the type of therapy. The target tissue may, for example, be neural tissue of the spinal cord, dorsal root, or dorsal root ganglion in accordance with some embodiments. In accordance with some examples, one or more respective electrodes in an electrode array of an electrical lead may perform functions of both signal delivery and signal sensing.

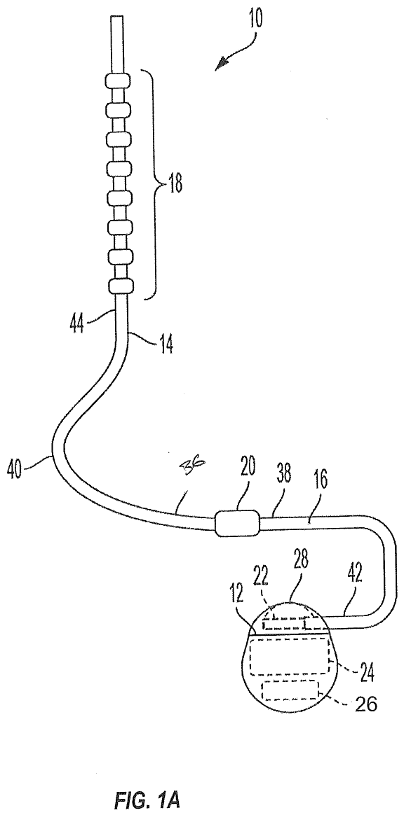

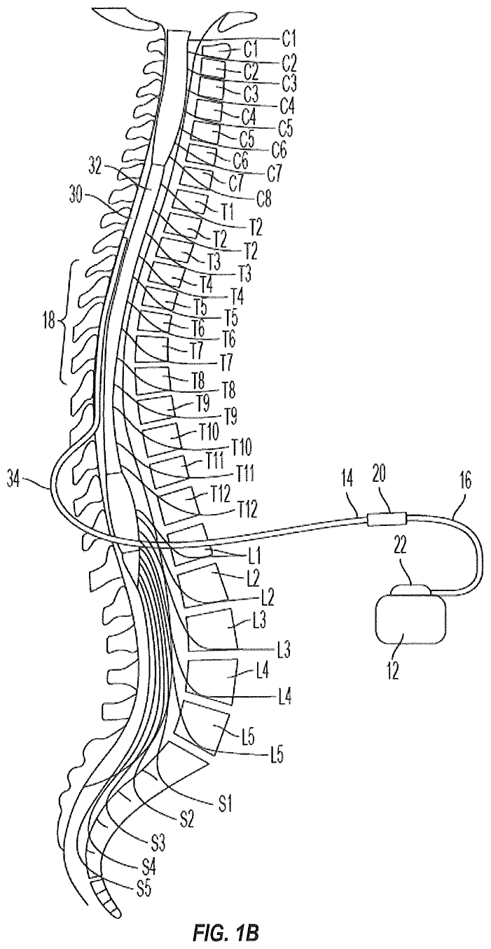

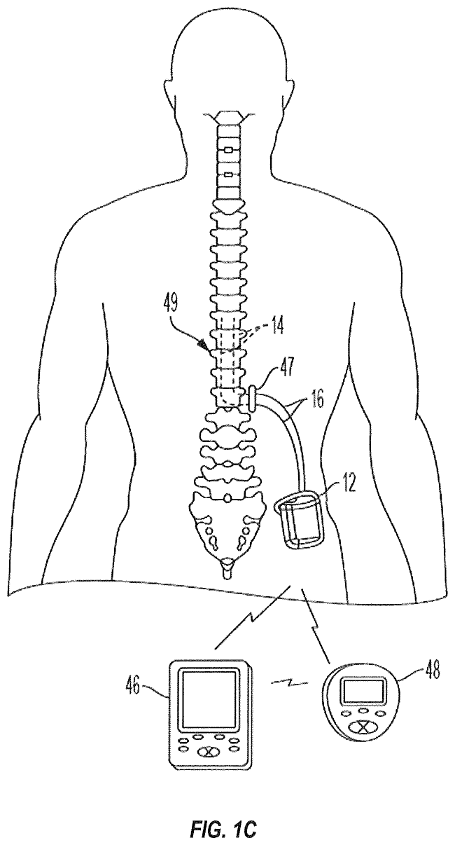

Referring now to FIG. A, depicted therein is a generalized neurostimulation system (NS) 10 that may be used in SCS, as well as other stimulation applications, that generates electrical pulses for application to target tissue of the patient. NS 10 generally includes implantable pulse generator 12, implantable lead 14, which carries an array of electrodes 18 (shown exaggerated for purposes of illustration), and optional implantable extension lead 16. Although only one lead 14 is shown, often two or more leads are used with electronic stimulus delivery systems (e.g., as shown in FIG. 1C), such as for implementing a multi-stim set in which the pulse generator rapidly switches between multiple stimulation programs providing stimulation pulses to the different leads.

Lead 14 includes elongated body 40 having proximal end 36 and distal end 44. Elongated body 40 typically has a diameter of between about 0.03 inches to 0.07 inches and a length within the range of 30 cm to 90 cm for spinal cord stimulation applications. Elongated body 40 may be composed of a suitable electrically insulative material, such as a polymer (e.g., polyurethane or silicone), and may be extruded as a unibody construction.

In the illustrated embodiment, proximal end 36 of lead 14 is electrically coupled to distal end 38 of extension lead 16 via a connector 20, typically associated with the extension lead 16. Proximal end 42 of extension lead 16 is electrically coupled to implantable pulse generator 12 via connector assembly 22 associated with housing 28. Alternatively, proximal end 36 of lead 14 can be electrically coupled directly to connector assembly 22.

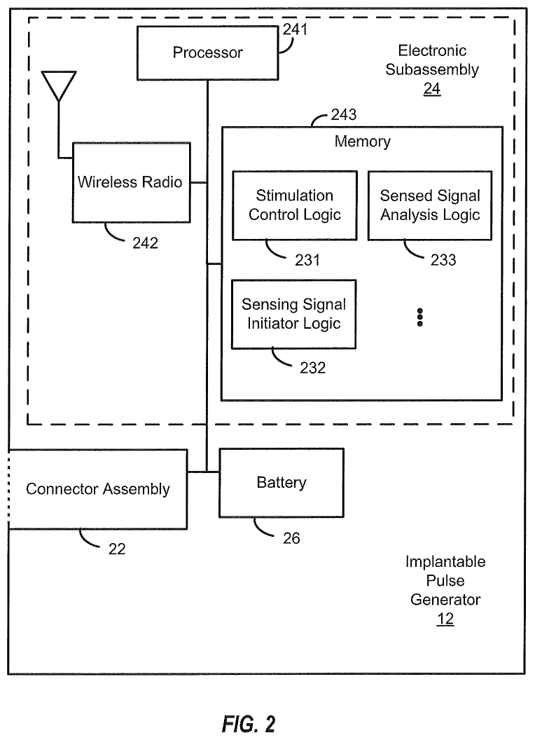

In the illustrated embodiment, implantable pulse generator 12 includes electronic subassembly 24 (shown as a dashed line box, further described in reference to FIG. 2 set forth below), which includes control and pulse generation circuitry (not shown) for delivering electrical stimulation energy to electrodes 18 of lead 14 in a controlled manner. Example sensing circuitry may include circuit elements including analog-to-digital converter (ADC) circuitry, amplifier circuitry, op-amp circuitry, bipolar transistors, and/or CMOS devices, the like. The sensing circuitry may include one or more circuit examples described herein according to some embodiments. Implantable pulse generator 12 of the illustrated embodiment may further include a power supply, such as battery 26.

Implantable pulse generator 12 provides a programmable stimulation signal (e.g., in the form of electrical pulses or substantially continuous-time signals) that is delivered to target stimulation sites by electrodes 18. In applications with more than one lead 14, implantable pulse generator 12 may provide the same or a different signal to electrodes 18 of the therapy delivery elements.

In accordance with some embodiments, implantable pulse generator 12 can take the form of an implantable receiver-stimulator in which the power source for powering the implanted receiver, as well as control circuitry to command the receiver-stimulator, are contained in an external controller inductively coupled to the receiver-stimulator via an inductive link. In another embodiment, implantable pulse generator 12 can take the form of an external trial stimulator (ETS), which has similar pulse generation circuitry as an implantable pulse generator (IPG), but differs in that it is a non-implantable device that is used on a trial basis after lead 14 has been implanted and prior to implantation of the IPG, to test the responsiveness of the stimulation that is to be provided.

Further, whereas some example embodiments herein may involve implantable devices, additional and/or alternative embodiments may involve external devices and/or noninvasive/minimally invasive (NIMI) devices (e.g., wearable biomedical devices, transcutaneous/subcutaneous devices, etc.) that may be configured to provide therapy to the patients roughly analogous to the implantable devices requiring appropriate stimulation and/or sense circuitry and protocols in some arrangements. Accordingly, all such devices may be broadly referred to as “medical devices”, “personal medical devices,” “personal biomedical instrumentation,” or terms of similar import, which may be controlled by an external controller device (e.g., operated by an authenticated patient, a clinician and/or their respective authorized agents), wherein suitable biosignals (e.g., nerve action potentials, which may be triggered in response to stimuli or otherwise) may be sensed, amplified and processed for purposes of some example embodiments of the present disclosure.

In some implantable and/or minimally/partially invasive arrangements, housing 28 may be composed of a biocompatible material, such as for example titanium, and forms a hermetically sealed compartment containing electronic subassembly 24 and battery 26 is protected from the body tissue and fluids. Connector assembly 22 is disposed in a portion of housing 28 that is, at least initially, not sealed. Connector assembly 22 carries a plurality of contacts that are electrically coupled with respective terminals at proximal ends of lead 14 or extension lead 16. Electrical conductors extend from connector assembly 22 and connect to electronic subassembly 24.

FIG. 1B illustrates lead 14 implanted in epidural space 30 of a patient in close proximity to the dura, the outer layer that surrounds spinal cord 32, to deliver the intended therapeutic effects of spinal cord electrical stimulation. The target stimulation sites may be anywhere along spinal cord 32. The target sites may, for example, include cervical, thoracic, lumbar, and sacral vertebral levels.

Because of the lack of space near lead exit point 34 where lead 14 exits the spinal column, implantable pulse generator 12 is generally implanted in a surgically-made pocket either in the abdomen or above the buttocks, such as illustrated in FIG. 1C. Implantable pulse generator 12 may also be implanted in other locations of the patient's body depending on the deployment scenario. Use of extension lead 16 facilitates locating implantable pulse generator 12 away from lead exit point 34. In some embodiments, extension lead 16 serves as a lead adapter if proximal end 36 of lead 14 is not compatible with connector assembly 22 of implantable pulse generator 12, since different manufacturers use different connectors at the ends of their stimulation leads and are not always compatible with connector assembly 22.

As illustrated in FIG. 1C, NS 10 also may include clinician programmer 46 and patient programmer 48. Clinician programmer 46 may be a handheld computing device that permits a clinician to program neurostimulation therapy for patient using input keys and a display. For example, using clinician programmer 46, the clinician may specify neurostimulation parameters for use in delivery of neurostimulation therapy. Clinician programmer 46 supports telemetry (e.g., radio frequency telemetry) with implantable pulse generator 12 to download neurostimulation parameters and, optionally, upload operational or physiological data stored by implantable pulse generator 12. In this manner, the clinician may periodically interrogate implantable pulse generator 12 to evaluate efficacy and, if necessary, modify the stimulation parameters.

Similar to clinician programmer 46, patient programmer 48 may be a handheld computing device. Patient programmer 48 may also include a display and input keys to allow patient to interact with patient programmer 48 and implantable pulse generator 12. Patient programmer 48 provides a patient with an interface for control of neurostimulation therapy provided by implantable pulse generator 12. For example, a patient may use patient programmer 48 to start, stop or adjust neurostimulation therapy. In particular, patient programmer 48 may permit a patient to adjust stimulation parameters such as duration, amplitude, pulse width and pulse rate, within an adjustment range specified by the clinician via clinician programmer 48, or select from a library of stored stimulation therapy programs.

Implantable pulse generator 12, clinician programmer 46, and patient programmer 48 may communicate via cables or a wireless communication. Clinician programmer 46 and patient programmer 48 may, for example, communicate via wireless communication with implantable pulse generator 12 using radio frequency (RF) telemetry techniques known in the art. Clinician programmer 46 and patient programmer 48 also may communicate with each other using any of a variety of local wireless communication techniques, such as RF communication according to the 802.11 or BLUETOOTH specification sets, infrared communication (e.g., according to the IrDA standard), or other standard or proprietary telemetry protocols.

Since implantable pulse generator 12 is located remotely from target location 49 for therapy, lead 14 and/or extension leads 16 is typically routed through pathways subcutaneously formed along the torso of the patient to a subcutaneous pocket where implantable pulse generator 12 is located. As used hereinafter, “lead” and “lead extension” are used interchangeably, unless the particular context clearly dictates otherwise.

Leads are typically fixed in place near the location selected by the clinician using one or more anchors 47, such as in the epidural space 30. Anchor 47 can be positioned on lead 14 in a wide variety of locations and orientations to accommodate individual anatomical differences and the preferences of the clinician. Anchor 47 may then be affixed to tissue using fasteners, such as for example, one or more sutures, staples, screws, or other fixation devices. The tissue to which anchor 47 is affixed may include subcutaneous fascia layer, bone, or some other type of tissue. Securing anchor 47 to tissue in this manner prevents or reduces the chance that lead 14 will become dislodged or will migrate in an undesired manner.

NS 10 may be operated to controllably deliver electrical pulses or signals to targeted tissue or nerves within a patient, such as for the treatment of one or more indications. Additionally, NS 10 may be operated to sense and/or analyze signals responsive to the electronic stimuli, such as to inform fiber recruitment, to implement closed-loop feedback control of electrical pulse delivery, etc. Accordingly, electronic subassembly 24 of implantable pulse generator 12 may include processors, electronics devices, hardware devices, electronics components, logical circuits, memories, software codes, firmware codes, etc., or any combination thereof configured for controlled stimulation and/or sensing operation. One or more functional blocks of electronic subassembly 24 may, for example, be implemented as discrete gate or transistor logic, discrete hardware components, or combinations thereof configured to provide logic for performing the functions described herein. Additionally or alternatively, when implemented in software, one or more functional blocks of electronic subassembly 24, or some portion thereof, may comprise code segments (e.g., one or more instruction sets, program code, programs, applications, etc.) operable upon a processor (e.g., a processing unit having computer readable media, such as a semiconductor memory device, a read only memory (ROM), a flash memory, an erasable ROM (EROM), etc., storing instructions which when executed perform functionality described herein) to provide logic for preforming the functions described herein. Processors utilized in implementing functions herein may, for example, comprise a general-purpose processor, a digital signal processor (DSP), an application specific integrated circuit (ASIC), a field programmable gate array (FPGA), or combinations thereof.

An example embodiment of implantable pulse generator 12 is illustrated in the block diagram of FIG. 2, wherein further details of exemplary electronic subassembly 24 are shown. Electronic subassembly 24 is shown in the example of FIG. 2 as being in communication with connector assembly 22 and battery 26, which may operate as described above with reference to FIGS. 1A-1C. Electronic subassembly 24 is further shown as including processor 241 in communication with wireless radio 242 and memory 243.

Wireless radio 242 may of embodiments may operate to facilitate wireless communication between implantable pulse generator 12 and one or more devices external thereto. For example, clinician programmer 46 and/or patient programmer 48 (FIG. 1C) may communicate with implantable pulse generator 12 via wireless radio 242. Wireless radio 242 may comprise an RF transceiver operable according to one or more wireless communication protocols (e.g., the 802.11 or BLUETOOTH specification sets, other standard or proprietary telemetry protocols, etc.).

Memory 243 of the example embodiment is operable to store various code segments executable by processor 241 to perform functions described herein. In particular, the code segments of the example in FIG. 2 includes stimulation control logic 231, sensing signal initiator logic 232, and sensed signal analysis logic 233. Stimulation control logic 231 may, for example, provide logic which when executed by processor 241 controls delivery of stimulation pulses to neural tissue via output of connector assembly 22 to lead 14 (FIGS. 1A-1C) according to a paresthesia-free stimulation regimen (e.g., high-frequency spinal cord stimulation (HFSCS) or burst stimulation), a conventional stimulation regimen, etc. Sensing signal initiator logic 232 of embodiments provides logic which when executed by processor 241 facilitates sensing responsive signals with respect to the application of the stimuli, such as through controlling operation to evoke responsive signals suitable for measurement and/or analysis. Sensed signal analysis logic 233 may, for example, provide logic which when executed by processor is operative to control sensing of signals, processing of sensed signals, analysis of sensed signals, and/or delivery of information (e.g., to stimulation control logic 231 executed by processor 241) regarding sensed signals. Memory 243 of embodiments may store code segments additionally or alternatively to those of the illustrated embodiment. For example, although not shown in the example of FIG. 2, memory 243 may store communication logic operable to control communication between implantable pulse generator 12 and one or more external systems (e.g., clinician programmer 46 and/or patient programmer 48), such as for receiving control signals and program code, transmitting data and telemetry, etc.

As described in further detail below, sensing signal initiator logic 232 of embodiments may operate to evoke responsive signals with sufficient signal strength and/or signal to noise (S/N) characteristics to reliably facilitate their measurement and/or analysis, even in situations where stimulation control logic 231 provides stimulation operation in accordance with a paresthesia-free stimulation regimen (e.g., HFSCS or burst stimulation). Sensed signal analysis logic 233 may thus be enabled to sense responsive signals having suitable characteristics for facilitating further processing and/or analysis, such as for informing fiber recruitment, providing information for closed-loop feedback control of the stimulus regimen by stimulation control logic 231, etc.

To aid in understanding concepts of the present invention facilitating operation as described above, examples with respect to implantable pulse generator 12 providing a burst stimulation regimen for SCS will be described. It should be appreciated, however, that concepts of the present invention may be applied with respect to various forms of paresthesia-free electrical stimulation (e.g., HFSCS, burst stimulation, high density stimulation, paresthesia-free noise stimulation, etc.) and/or for a variety of target areas (e.g., SCS, dorsal root stimulation, and dorsal root ganglion stimulation). For example, sensing signal stimulation according to some examples may be implemented with respect to a stimulation regimen which defines a high frequency stimulation pattern that is controlled with a duty cycle having on-periods and off-periods of stimulation, wherein sensing signal stimulation pinging-pulses are provided in association with off-cycles of the high frequency stimulation pattern.

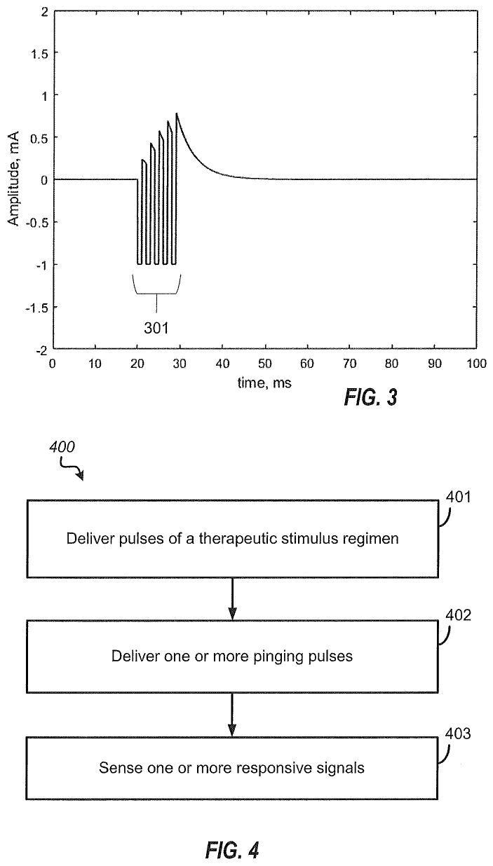

In operation according to an exemplary embodiment, implantable pulse generator 12 may implement a burst stimulation therapy to suppresses neuropathic pain without eliciting paresthesia (e.g., paresthesia-free stimulation). In operation according to a burst stimulation regimen, packets (e.g., “bursts”) of high-frequency impulses are delivered periodically at a current intensity below the paresthesia threshold. For example, a burst stimulation waveform may include five pulses of cathodic pulses (or anodic pulses at the anode) with 1000 μs pulse width each, as shown in FIG. 3 (wherein only a single burst is shown as burst 301). In a specific example, the frequency within the burst or pulse rate (the “intra-burst frequency”) may be set to be 500 Hz, such as for SCS applications. Other intra-burst frequencies may be employed according to some embodiments to optimize therapy for a given patient. Continuing with the specific example, the frequency at which the bursts repeat (the “inter-burst frequency”) may be nominally set to be 40 Hz, such as for SCS applications, and may be adjusted based on user preference and applications. It should be understood, however, that other intra-burst frequencies and/or inter-burst frequencies may be used, whether for SCS or other applications (e.g., the intra-burst frequency and/or nominal inter-burst frequency may be adjusted for dorsal root ganglion (DRG) stimulation).

In operation of implantable pulse generator 12, one or more signals (“responsive signals”) generated or otherwise present in response to the electrical stimulation pulses may be sensed, such as for use in analyzing fiber recruitment, adjusting or otherwise controlling one or more aspect of the burst stimulation regimen, etc. An evoked neural response may, for example, be constituted of evoked compound action potentials. Evoked compound action potentials (ECAPs) are an example of responsive signals which may be sensed, processed, analyzed, and/or used in providing closed-loop feedback according to embodiments of the invention.

ECAPs are signals evoked by electrical stimulations and recorded near a bundle of fibers. ECAPs usually arrive less than 1 ms (<1 ms) after a corresponding stimulation pulse and last in the range of approximately one half to one millisecond (0.5-1 ms). In the case of SCS, the fibers that generate ECAPs are sensory fibers located in the dorsal column. With enough populational activation, sensory Δβ fibers also induce paresthesia, and therefore are the primary fibers responsible for the mechanism of action for traditional tonic SCS (e.g., generating paresthesia from Δβ fibers to mask pain). ECAPs may be measured and analyzed, for example, to evaluate and/or control the comfort and efficacy of a SCS treatment regimen. However, ECAPs or similarly generated responsive signals having signal strength, signal to noise ratio (SNR), and/or other characteristics for their reliable measurement and analysis may not be present in some situations. For example, burst stimulation at clinical amplitudes may not activate a sufficient number of dorsal column fibers, and thus usually results in no measurable or no meaningful ECAP data from sensor circuitry of the SCS IPG.

One example solution to sensing ECAPs with respect to a paresthesia-free stimulation technique such as burst stimulation may be to increase the amplitude of the stimulation pulses of the paresthesia-free stimulation regime to beyond the level for perception and/or for generating paresthesia (e.g., burst stimulation pulses with amplitudes higher than 1.8 mA). Although this solution may be suitable in situations such as asleep implant procedures where patients do not have to experience the sensation with high stimulation pulse amplitudes, it is not well suited for general use of an implantable pulse generator to treat chronic pain of a patient. For example, the use of such increased amplitude of the pulses of otherwise paresthesia-free stimulation therapy to thereby increase the quality of the ECAP measurement and control process, would significantly reduce the patient's experience and relief of pain to the paresthesia-free stimulation therapy—essentially eliminating the paresthesia-free nature of the stimulation and likely modifying the mechanism of action.

Some embodiments of the invention utilize sensing signal initiator logic 232 in association with implementation of paresthesia-free stimulation by stimulation control logic 231 to implement sensing signal stimulation evoking responsive signals suitable for measurement and/or analysis by sensed signal analysis logic 233 without substantially changing the patient's paresthesia-free therapy into a paresthesia-based therapy. In operation according to some examples, sensing signal initiator logic 232 when executed by processor 241 may facilitate sensing of ECAPs in association with implementation of burst stimulation operating to provide paresthesia-free stimulation. Sensing signal initiator logic 232 of embodiments of the invention may, for example, control implantable pulse generator 12 to deliver one or more non-therapeutic pulses (“pinging-pulses”) configured for evoking responsive signals (e.g., ECAPs) suitable for measurement and/or analysis in association with the application of neural stimuli. In operation of sensing signal initiator logic implementing sensing signal stimulation of embodiments of the invention, pinging-pulses are provided for eliciting ECAPs and/or other responsive signals with respect to paresthesia-free stimulation (e.g., burst stimulation or high frequency stimulation) substantially without eliciting paresthesia either by the pinging-pulses or the therapeutic stimulation. For example, aspects of a pinging-pulse (e.g., current intensity amplitude, pulse width, etc.) and/or pinging-pulse duty cycle (e.g., frequency of pinging-pulses, aggregate current intensity amplitude(s), aggregate pulse width(s), etc.) may be configured to avoid eliciting paresthesia in a patient.

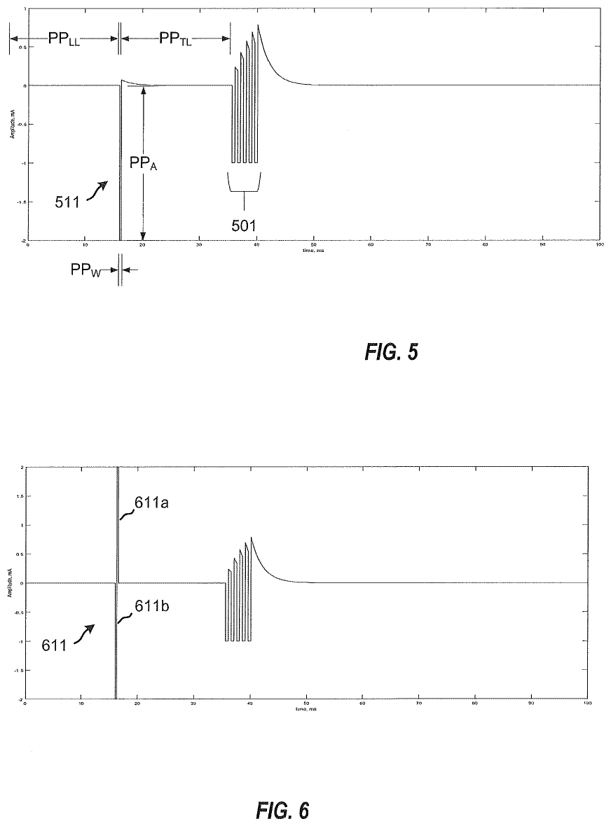

FIG. 4 shows an example flow according to a process operable to evoke responsive signals suitable for measurement and/or analysis in association with the application of therapeutic neural stimuli in accordance with concepts of the present disclosure. That is, flow 400 shown in FIG. 4 provides a process to evoke sensing signals in association with corresponding therapeutic neural stimuli. The functions of flow 400 shown in FIG. 4 may, for example, be performed by an embodiment of implantable pulse generator 12, such as through operation of processor 241 executing stimulation control logic 231, sensing signal initiator logic 232, sensed signal analysis logic 233, and/or other logic for performing functions as described.

At block 401 of exemplary flow 400, pulses of a therapeutic stimulus regimen are delivered by implantable pulse generator 12, such as to target tissue within a patient via electrodes 18 of lead 14. For example, processor 241 may execute stimulation control logic 231 to provide and control delivery of the pulses of the therapeutic stimulus regimen. The amplitudes of the pulses of the therapeutic pulses of the stimulation regimen may be constant or may vary, such as according to the treatment being delivered, the particular patient being treated, etc.

The therapeutic stimulus regimen may comprise one or more a paresthesia-free stimulation regimen (e.g., high-frequency stimulation or burst stimulation), etc. In accordance with some examples, the therapeutic stimulus regimen may comprise a paresthesia-free stimulation regimen which itself results in no ECAPs or results in ECAPs of such low signal strength and/or SNR as to make their measurement and/or analysis impractical or even impossible. As a specific example, the therapeutic stimulus regimen may delivery a burst stimulation configuration of pulses, such as shown in the example of FIG. 3.

Flow 400 of the illustrated embodiment is operable to evoke responsive signals suitable for measurement and/or analysis in association with the application of the therapeutic stimulus regimen. Accordingly, at block 402, one or more pinging-pulses are delivered by implantable pulse generator 12, such as to the target tissue within the patient via electrodes 18 of lead 14. For example, processor 241 may execute sensing signal initiator logic 232 to provide and control delivery of the pinging-pulses of the sensing signal stimulation. Pinging-pulses utilized to initiate sensing signals of embodiments of the invention comprise non-therapeutic signals configured for evoking responsive signals suitable for measurement and/or analysis in association with the therapeutic stimulus regimen. Pinging-pulses may, for example, be provided for facilitating sensing ECAPs in association with operation of a paresthesia-free stimulation technique. According to some examples, the pinging-pulses may be provided for eliciting ECAPs with respect to burst stimulation, without eliciting paresthesia either by the pinging-pulses or the burst stimulation. In operation according to embodiments, the IPG (e.g., sensed signal analysis logic 233) may monitor a signal quality (e.g., a SNR) of the evoked neural response and control an amplitude level of the pinging-pulses in response to the quality level of the evoked neural response.

In operation at block 402 of embodiments of the invention, sensing signal initiator logic 232 may provide for an interleaved implementation to introduce the one or more pinging-pulses in between groups of pulses of the therapeutic stimulus regimen (e.g. between burst groups of a burst stimulation regimen, during a pause of appropriate duration between instances of a high frequency tonic stimulation regimen, etc.). The pinging-pulses of an interleaved implementation may, for example, comprise monophasic cathodic pulses, biphasic charge-balanced cathodic pulses (e.g., with passive or active discharge), anodic-leading actively charge-balanced pulses, or any combination thereof.

FIG. 5 shows an example of a pinging-pulse (e.g., a cathodic pulse with passive discharge) interleaved with respect to bursts of a burst stimulation regimen. In particular, pinging-pulse 511 is shown provided within between bursts of a burst stimulation regimen (only burst 501 being shown, and it being understood that another burst having the same or different burst stimulation waveform precedes pinging-pulse 511 in the example timeline). Burst 501 may, for example, comprise a burst stimulation waveform corresponding to that of FIG. 3 described above. In operation of an interleaved pinging-pulse implementation of some examples, measuring an evoked neural response in a patient in response to the pinging-pulses occurs without including an evoked neural response to therapeutic pulses of the stimulation program.

Pinging-pulses of embodiments of an interleaved implementation may be configured in some implementations to evoke responsive signals (e.g., sensing signals) suitable for measurement and/or analysis in association with the therapeutic stimulus regimen without eliciting paresthesia. For example, various aspects of a pinging-pulse, such as one or more of pulse width, amplitude, latency between the pinging-pulse and therapeutic pulses, active discharge pulse width, anodic-leading pulse width, etc., may be selected for evoking a sensing signal without eliciting paresthesia.

According to some embodiments of an interleaved implementation of pinging-pulses, the cathodic phase of a pinging-pulse is controlled to be in the range of 60-1000 μs in pulse width (e.g., 60 μs≤PP_{W}≤1000 μs). The amplitude of the cathodic phase is selected and/or adjusted in operation according to embodiments such that a single pinging-pulse evokes one or more sensing signals (e.g., eliciting ECAPs in the dorsal column in the case of SCS). In some examples, the pinging-pulse amplitude is selected in the range of 0.5-5 mA (e.g., 0.5 mA≤PP_{A}≤5 mA). The pinging-pulse amplitude may, for example, be selected in part based upon various aspects of the particular implementation, such as pinging-pulse width, implant location, etc. A pinging-pulse trailing latency of at least 1.2 ms (e.g., PP_{TL}≥1.2 ms) is provided between a pinging-pulse of an interleaved implementation of embodiments and the subsequent therapeutic pulses (e.g., the pulses of burst 501), such as to facilitate sufficient time for sensing responsive signals (e.g., ECAPs). A pinging-pulse leading latency may be based upon the intra-burst frequency of the therapeutic pulses, the pinging-pulse trailing latency, and the pinging-pulse pulse width.

Pinging-pulses of embodiments of an interleaved implementation may not be present at every interval between therapeutic pulses (e.g., a pinging-pulse may not be delivered in every inter-burst-interval whereby the frequency or duty cycle of the pinging-pulses is less than the inter-burst rate of the burst stimulation pattern). In accordance with some examples, the IPG generates the pinging-pulses at a frequency or duty cycle that is selected to be sufficiently low to prevent the pinging-pulses from generating paresthesia in the patient at an amplitude level selected to evoke a neural response for measurement by the IPG. For example, some embodiments of the invention may distribute the occurrences of pinging-pulse (e.g., maintaining the inter-pinging-pulse frequency at low rate, such as 20 Hz or lower) in order to avoid or minimize resulting paresthesia. The frequency of occurrence of the pinging-pulses may be set by a clinician during a programming process to verify the pinging-pulses do not generate paresthesia in a given patient according to some embodiments.

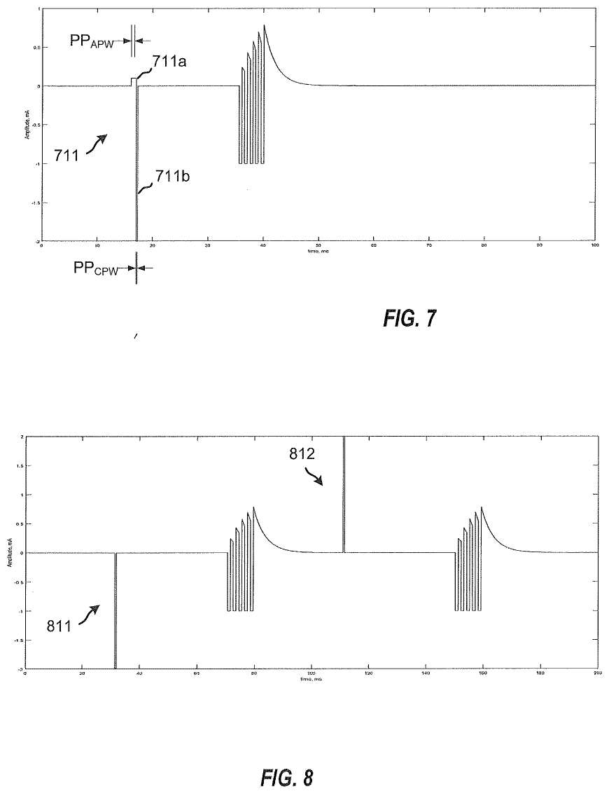

According to some embodiments of an interleaved implementation of pinging-pulses, a pinging-pulse may be provided with active discharge. FIG. 6 shows an example of a pinging-pulse with active discharge inserted between burst stimulation pulses (the preceding burst of which is not visible in the graph of FIG. 6). In the example of FIG. 6, pinging-pulse 611 includes cathodic phase 611a and anodic phase 611b (e.g., generating pairs of pulses in sequence for the pinging-pulses that have opposite polarity), wherein one of the phases may provide active discharge with respect to the other phase of the phase pair (e.g., anodic phase 611b providing an active discharge with respect to cathodic phase 611a). In operation according to embodiments in which a pinging-pulse is actively discharged, the active discharge phase (e.g., anodic phase 611b in the example of FIG. 6) preferably matches the pulse width of the leading phase of the pinging-pulse (e.g., cathodic phase 611a in the example of FIG. 6).

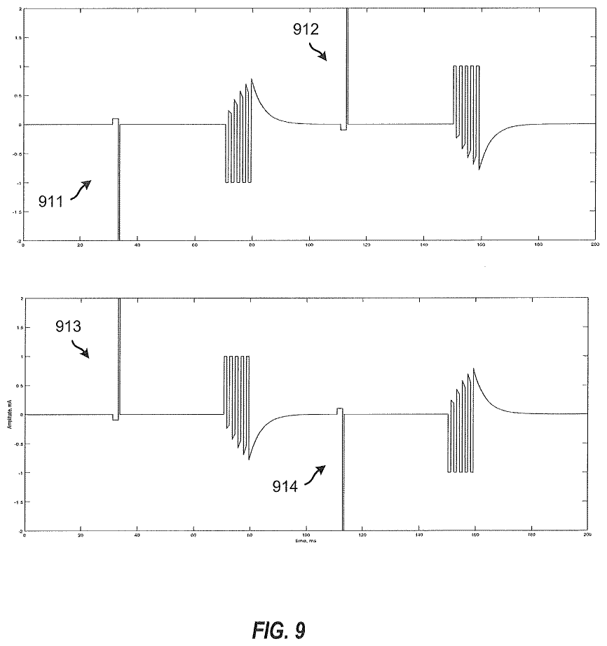

Although the foregoing examples of interleaved pinging-pulses have been set forth with reference to pinging-pulse instances having a cathodic leading phase, it should be appreciated that pinging-pulses having an anodic leading phase may be utilized in addition to or in the alternative to pinging-pulses having a cathodic leading phase. FIG. 7 shows an example of an anodic-leading pinging-pulse with active discharge was inserted between burst stimulation pulses (the preceding burst of which is not visible in the graph of FIG. 7). In the example of FIG. 7, pinging-pulse 711 includes anodic phase 711a and cathodic phase 711b, wherein cathodic phase 711b provides active discharge with respect to anodic phase 711a. When a pinging-pulse is implemented with an anodic-leading pulse according to some embodiments of the invention, the anodic pulse width (APW) may range from 500-1000 μs (e.g., 500 μs≤PP_{APW}≤1000 μs), and the cathodic pulse width (CPW) is preferably less than 200 μs (e.g., PP_{CPW}<200 μs). Such a configuration of anodic leading pinging-pulse may facilitate the cathodic phase eliciting sensing signals (e.g., responsive signals in the form of ECAPs) following the anodic phase. In operation according to embodiments of an anodic leading pinging-pulse configuration, the anodic phase provides a preconditioning pulse that increases the excitability of Δβ fibers, and the trailing cathodic pulse has higher amplitude than anodic phase pulse. The anodic and cathodic pulses can be charge balanced or slighted charge imbalanced (e.g., the charge resulting from the amplitude and pulse width of the pinging-pulse anodic phase is equal to or approximately equal to the charge resulting from the amplitude and pulse width of the pinging-pulse cathodic phase).

The pinging-pulses of a sequence of pinging-pulses of an interleaved implementation may each be configured the same or one or more may be configured differently. For example, interleaved pinging-pulses may be provided in an implementation in which the pinging-pulses may switch between cathodal mode/polarity and anodal mode/polarity within a stimulation train. FIG. 8 shows an example of a pulse train in which pinging-pulses of alternating polarity are provided. In particular, pinging-pulse 811 of the example comprises a cathodic monophasic pulse, whereas pinging-pulse 812 comprises an anodic monophasic pulse. Such an implementation is particularly well suited for implementing monophasic pinging-pulses because the alternating pulses cancel each other. Alternating the polarity of pinging-pulses, such as shown in the example of FIG. 8, may be utilized according to embodiments of the invention to improve the SNR of elicited sensing signals. For example, the alternation in polarity may be utilized to improve the SNR of ECAPs because the opposite polarity of stim pulses can be added to zero, and the ECAPS themselves can be averaged. Such an interleaved implementation of pinging-pulses with alternating polarity allows the use of a single pulse train to improve ECAP SNR, rather than two separate pulse trains with flipped polarity of electrodes to summate to improve ECAP SNR (e.g., conducting a summation operation of the respective evoked neural response to both pulses in sequence of opposite polarity to increase signal-to-noise ratio), and therefore may reduce experimenting time by half. Additionally, the frequency of the pinging pulses may be adjusted. For example, the frequency may be controlled such that only one pinging pulses is provided between bursts for some embodiments. Lower frequency pinging pulses may be applied such that a pinging pulses are not generated before every burst but only for a subset of bursts in the overall waveform pattern. Alternatively, higher frequencies may be selected for the pinging pulses may be selected such that more than one pining pulse is provided between consecutive bursts of pulses for other embodiments.

Interleaved implementations of pinging-pulses may be utilized with multi-stim sets according to embodiments of the invention. For example, interleaved pinging-pulses may be implemented via a multi-stim set in which the implantable pulse generator operates to rapidly switch between two programs of opposite polarity between electrodes. FIG. 9 shows an example of a multi-stim pulse train of two electrodes in which pinging-pulses of alternating polarities are interleaved. In particular, pinging-pulses 911 and 912 of alternating polarities in the illustrated example are interleaved in the pulse train of a first electrode and pinging-pulses 913 and 914 of alternating polarities in the example are interleaved in the pulse train of a second electrode. In addition to the pinging-pulses alternating in polarity, the therapeutic pulse groups themselves are also alternated in polarity.

In addition or in alternative to providing for an interleaved pinging-pulse implementation, operation at block 402 of embodiments of the invention may include sensing signal initiator logic 232 providing for a postfixed implementation to introduce the one or more pinging-pulses with respect to pulses of the therapeutic stimulus regimen (e.g. appended to burst groups of a burst stimulation regimen, appended to a pulse train of a high frequency tonic stimulation regimen, etc.). The pinging-pulses of a postfixed implementation may, for example, comprise pulse configurations based upon or corresponding to pulses of the therapeutic stimulus regimen.

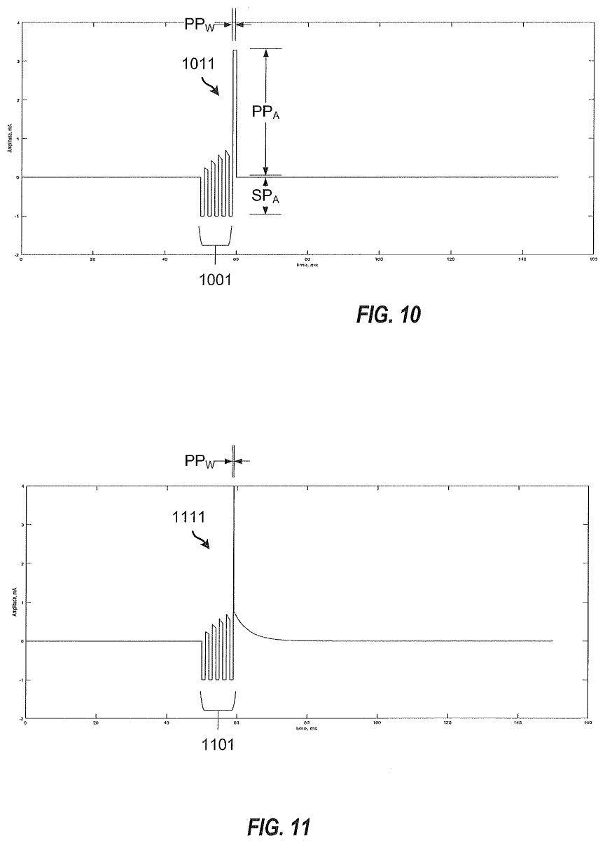

FIG. 10 shows an example of a pinging-pulse (e.g., an anodic charge-balanced active discharge pulse) appended or postfixed to a group of therapeutic pulses. In particular, burst 1001 of a burst stimulation regimen is shown as modified to include pinging-pulse 1011 optimized for eliciting sensing signals (e.g., ECAPs). In accordance with some examples, the last passive discharge phase of a burst stimulation waveform of the burst stimulation regimen may be replaced with a pinging-pulse of embodiments of the invention. In the example of FIG. 10, an otherwise last passive discharge phase of burst 1001 has been replaced with pinging-pulse 1011 providing an active charge-balancing anodic pulse. Burst 1001 may, for example, comprise a modified burst stimulation waveform corresponding to that of FIG. 3 described above, wherein the first four bursts should have properties of the burst stimulation waveform of burst 301 and the last pulse of the burst comprises a cathodic pulse followed by an anodic pulse. Accordingly, the last passive discharge of the burst stimulation waveform of burst 301 is replaced by an active discharge in an example of burst 1001.

Pinging-pulses of embodiments of a postfixed implementation are configured to evoke responsive signals (e.g., sensing signals) suitable for measurement and/or analysis in association with the therapeutic stimulus regimen without eliciting paresthesia. For example, various aspects of a pinging-pulse, such as one or more of pulse width, amplitude, correspondence to therapeutic pulse train, etc., may be selected for invoking a sensing signal without eliciting paresthesia.

According to embodiments of a postfixed implementation of pinging-pulses, the amplitude (PP_{A}) of the anodic phase of a pinging-pulse provided with respect to a burst may be determined by calculating the total remaining charge of previous full burst groups, and dividing the charge by the pulse width of the anodic pulse. The pinging-pulse amplitude (e.g., anodic phase amplitude) of embodiments may range from 3 to 50 times the amplitude of the therapeutic stimulation pulse (e.g., cathodic phase amplitude), such as depending upon the impedance of the electrode-tissue interface and the anodic pulse width (e.g., PP_{A} may range from 3(SP_{A}) to 5(SP_{A})). In accordance with embodiments of a postfixed implementation, the pinging-pulse amplitude may be capped at the discomfort amplitude (e.g., PP_{A}<a comfort threshold) so that the patient does not feel discomfort.

Pinging-pulses of postfixed implementations of embodiments of the invention may comprise relatively small pulse widths (e.g., 100 μs pulse width, as compared to a more common 1000 μs pulse width of a therapeutic stimulation pulse). In accordance with some embodiments, the amplitude of pinging-pulses having a small pulse width may be correspondingly capped to ensure safety. In the use of such small pulse width pinging-pulses having capped amplitudes, charge may remain that is not completely balanced. Accordingly, the implantable pulse generator may, according to some embodiments, proceed to discharge the remaining charges with passive discharge. FIG. 11 shows burst 1101 of a postfixed implementation for eliciting sensing signals (e.g., ECAPs), wherein the last passive discharge phase of the burst has been replaced with pinging-pulse 1111 comprising an active charge-balancing anodic pulse having a small pulse width. As shown in FIG. 11, the anodic pulse width of pinging-pulse 1111 is small, and the amplitude is capped, whereby passive discharge is used to discharge remaining charges. In operation according to some embodiments, a delay (e.g., trailing latency) of at least 1.2 ms (e.g., PP_{TL}≥1.2 ms) may be provided between the pinging-pulse and the passive discharge to facilitate sensing of responsive signals (e.g., ECAPs).

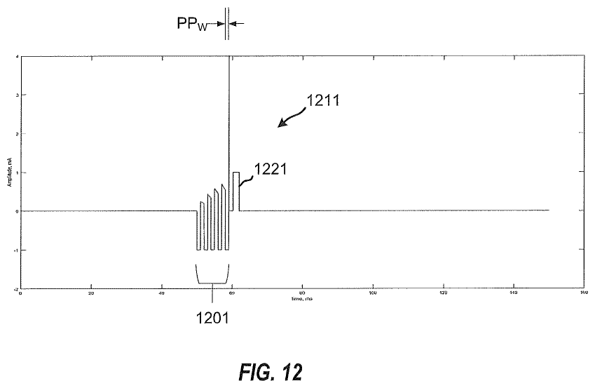

According to embodiments of a postfixed implementation of pinging-pulses, an active pulse may be provided to balance out the charges. For example, another anodic pulse of equal amplitude to that of the cathodic burst pulses may be added after the anodic pulse of a postfixed pinging-pulse. The pulse width of such as charge balancing active pulse may be calculated based on a duration to completely or substantially balance out the remaining charge. According to some examples, a trailing latency (e.g., PP_{TL}≥1.2 ms) may be provided following the pinging-pulse and before initiation of the charge balancing active pulse, such as to facilitate sensing of responsive signals (e.g., ECAPs). FIG. 12 shows burst 1201 of a postfixed implementation for eliciting sensing signals (e.g., ECAPs), wherein the last passive discharge phase of the burst has been replaced with pinging-pulse 1211 comprising an active charge-balancing anodic pulse having a small pulse width. As shown in FIG. 12, the anodic pulse width of pinging-pulse 1211 is small, and the amplitude is capped, whereby charge balancing pulse 1221 is used to implemented to balance out the charge (e.g., following a latency delay, PP_{TL}).

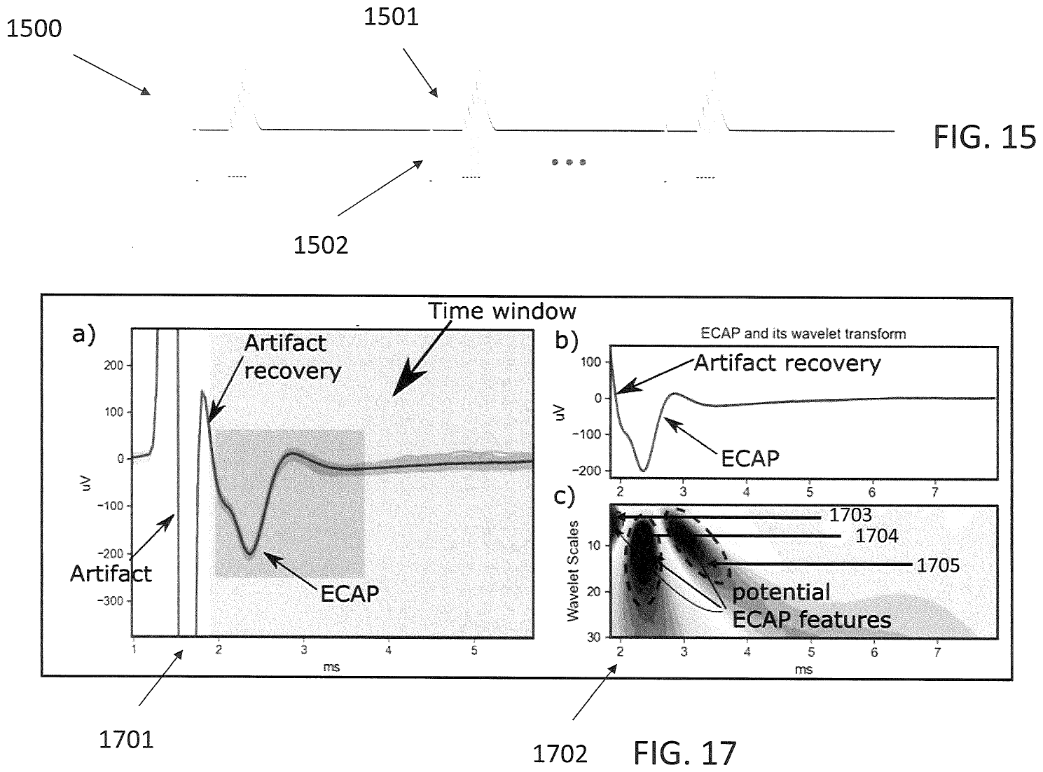

In other embodiments, the first pulse of a burst in a stimulation pattern may be modified to promote ECAP sensing. As shown in FIG. 15, waveform pattern 1500 includes multiple bursts of pulses generated in succession including burst 1501. As shown, burst 1501 (and other burst in the pattern) includes a delay between the first pulse and the second pulse that is longer than the delay between other pulses in burst 1501. This longer delay facilitates the sensing of the ECAP response to the first pulse of burst 1501. The delay between the first pulse and the second pulse may be a programmable setting to optimize ECAP sensing for a specific patient (either by clinician programming or automatically by sensing circuitry and ECAP signal analysis). The implementation of burst 1501 differs from the use of tonic pinging pulses in that no passive discharge occurs between the first pulse of burst 1501 and the second pulse of 1502. In some embodiments, the amplitude and/or pulse width of the first pulse may also be increased or modified to facilitate the sensing of the ECAP response thereto.

Pinging-pulses of embodiments of a postfixed implementation may not be present with respect to every group of therapeutic pulses (e.g., a pinging-pulse may not be delivered in every inter-burst-interval). In accordance with some examples, the IPG may generate the pinging-pulses at a frequency or duty cycle that is selected to be sufficiently low to prevent the pinging-pulses from generating paresthesia in the patient at an amplitude level selected to evoke a neural response for measurement by the IPG For example, embodiments of the invention may distribute the occurrences of pinging-pulse (e.g., maintaining the inter-pinging-pulse frequency at low rate, such as 20 Hz or lower) in order to avoid or minimize resulting paresthesia. The frequency of the pinging-pulses may be set by a clinician during a programming procedure to verify that the pinging-pulses do not elicit paresthesia in a given patient.

Postfixed implementations of pinging-pulses may be utilized with multi-stim sets according to embodiments of the invention. For example, similar to the interleaved pinging-pulses implemented with respect to the multi-stim pulse train of FIG. 9 discussed above, postfixed pinging-pulses of alternating polarities may be implemented with respect to a multi-stim pulse train of two electrodes according to some embodiments.

Having described examples of operation at block 402 of FIG. 4 to evoke responsive signals suitable for measurement and/or analysis in association with the application of the therapeutic stimulus regimen, and continuing with the description of flow 400, one or more responsive signals may be sensed at block 403. For example, sensing signals (e.g., ECAPs) elicited by a pinging-pulse (e.g., an instance of an interleaved pinging-pulse or a postfixed pinging-pulse) may be monitored, received, etc. by implantable pulse generator 12, such as via electrodes 18 of lead 14. In operation according to embodiments, processor 241 may execute sensed signal analysis logic 233 for monitoring sensing signals present in response to a pinging-pulse delivered by the implantable pulse generator.

Sensing signals monitored according to embodiments of the invention may be utilized in a number of ways. For example, sensed signal analysis logic 233 of embodiments may perform processing of sensing signals elicited by pinging-pulses to derive various attributes of the monitored sensing signals, such as for providing to a user (e.g., clinician), determining fiber recruitment, implementing changes to a corresponding therapeutic stimulation regimen, etc.

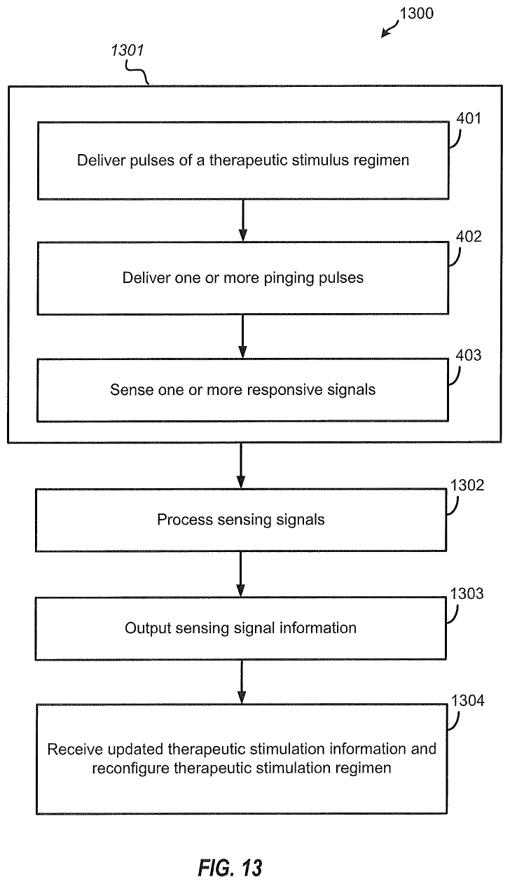

FIG. 13 shows an example process in which sensing signals elicited by pinging-pulses of an embodiment of the invention are utilized in an open-loop implementation for configuration of a therapeutic stimulation regimen. Block 1301 of flow 1300 shown in FIG. 13 comprises a process to elicit sensing signals in association with corresponding therapeutic neural stimuli corresponding to embodiments of flow 400 described above.

As an example of operation at block 1301 of some embodiments, SCS may be provided to a patient using an IPG. The operation of this example may include selecting one or more parameters for a stimulation program for SCS to provide electrical pulses to the patient without generating paresthesia in the patient. The selecting may comprise selecting a first amplitude value to control respective pulse amplitudes of therapeutic pulses of the stimulation program. The operation may also include generating, by the IPG, electrical pulses for the stimulation program according to the one or more parameters, and generating, by the IPG, pinging-pulses at amplitudes greater than pulse amplitudes of the therapeutic pulses of the stimulation program. The pinging-pulses may be interleaved with therapeutic pulses of the stimulation program. The operation may further include applying the electrical pulses generated for the stimulation program and the pinging-pulses to neural tissue of the spinal cord without generating paresthesia in the patient, as well as measuring an evoked neural response in the patient in response to the pinging-pulses.

In another example of operation at block 1301 of some embodiments, SCS may be provided to a patient using an IPG. The operation of this example may include selecting one or more parameters for a stimulation program for SCS to provide electrical pulses to the patient without generating paresthesia in the patient. The operation may also include generating, by the IPG, electrical pulses for the stimulation program according to the one or more parameters. The generating electrical pulses for the stimulation program may comprise modifying a pulse amplitude of selected pulses for the stimulation program by increasing the pulse amplitude to a level for accurate measurement of a neural response by the IPG. The selected pulses may, for example, constitute twenty percent or less of a total number of pulses generated for the stimulation program. The operation may further include applying the electrical pulses generated for the stimulation program to neural tissue of the spinal cord without generating paresthesia in the patient, and measuring, by the IPG, an evoked neural response in the patient in response to pulses of stimulation program with increased pulse amplitude for accurate measurement by the IPG.

At block 1302 of the example embodiment, monitored sensing signals are processed for obtaining various information useful with respect to configuration/reconfiguration of a corresponding therapeutic stimulation regimen. For example, sensed signal analysis logic 233 of embodiments may analyze one or more sensing signals (e.g., ECAPs) to determine whether the energy content in various frequency clusters of a sensing signal is within an acceptable range (e.g., performing threshold analysis using one or more thresholds, such as a recruitment threshold, comfort threshold, paresthesia threshold, etc., representing a selected neural stimuli profile).

Correspondingly, at block 1303, sensing signal information resulting from the monitoring and processing of sensing signals may be output by the implantable pulse generator. For example, sensed signal analysis logic 233 may utilize wireless radio 243 to communicate various information with respect to one or more sensing signal, such as information indicating aspects of the effect of the stimulus regimen (e.g., that no pain or an acceptable low level of pain is experienced by the patient, that no paresthesia or an acceptably low level of paresthesia is experienced by the patient, etc.), to an external device (e.g., clinician programmer 46 of FIG. 1C).

At block 1304 of the illustrated embodiment, updated therapeutic stimulation information is received by the implantable pulse generator and the therapeutic stimulation regimen updated accordingly. For example, a clinician may refer to the sensing signal information for making one or more adjustments to neural stimuli of the therapeutic stimulation regimen, such as using clinician programmer 46. Thereafter, updated therapeutic stimulation information comprising the adjustments may be provided to stimulation control logic 231 via wireless radio 242 such that stimulation control logic 231 may reconfigure the therapeutic stimulation regimen and implement a thusly updated therapeutic stimulation regimen.

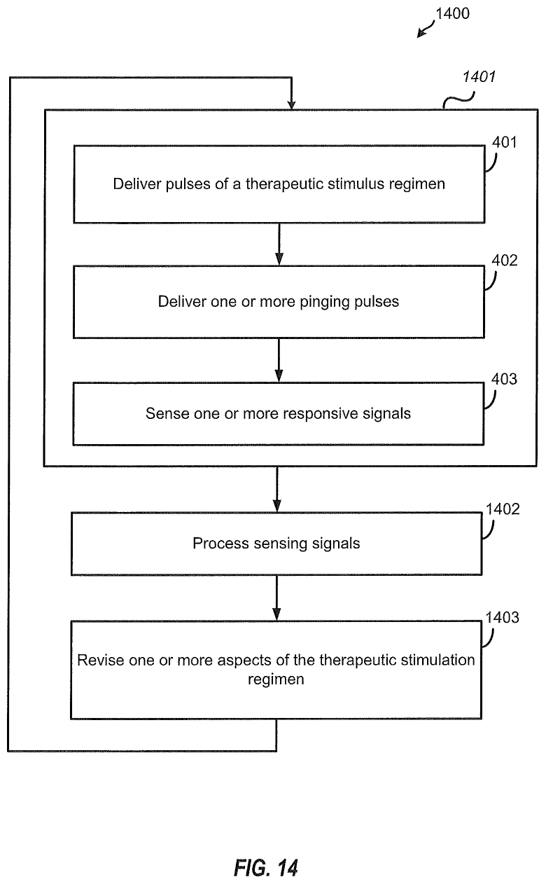

FIG. 14 shows an example process in which sensing signals elicited by pinging-pulses of an embodiment of the invention are utilized in a closed-loop implementation for configuration of a therapeutic stimulation regimen. Block 1401 of flow 1400 shown in FIG. 14 comprises a process to elicit sensing signals in association with corresponding therapeutic neural stimuli corresponding to embodiments of flow 400 described above.

At block 1402 of the illustrated embodiment, monitored sensing signals are processed for identifying candidate updated therapeutic stimulation waveforms. For example, sensed signal analysis logic 233 of embodiments may provide processing to convert one or more sensing signals (e.g., ECAPs) to the frequency domain (e.g., fast Fourier transform) and implement various analysis techniques, such as frequency discrimination, profile analysis, etc., to derive activity data useful in configuring/reconfiguring one or more aspect of a corresponding therapeutic stimulation regimen. In operation according to embodiments, sensed signal analysis logic 233 may analyze one or more features from a morphology of sensing signals over time, sum the occurrences of one or more features that occur with respect to sensing signals over a period of time, etc., for generating the activity data. Activity data generated through analysis of the sensing signals may be used in determining aspects of updated therapeutic stimulation waveforms.

Correspondingly, at block 1403, results of the processing and analysis of the sensing signals is utilized to revise one or more aspects of the therapeutic stimulation regimen. For example, sensed signal analysis logic 233 may provide updated therapeutic stimulation information, such as may be revised based upon activity data generated from the sensed signals, to stimulation control logic 231. Operation according to flow 1400 of some examples may thus detect a change in the evoked neural response of the patient to the pinging-pulses and automatically adjust one or more parameters for the stimulation program (e.g., modifying an amplitude level for respective pulses generated for the stimulation program without generating paresthesia in the patient) in response to detecting the change. Accordingly, stimulation control logic 231 may reconfigure the therapeutic stimulation regimen and implement the updated therapeutic stimulation regimen (e.g., returning to block 1401).

As can be appreciated from the foregoing, sensing signal stimulation implementations of embodiments of the invention evoke responsive signals with sufficient signal strength and/or S/N characteristics to provide sensing signals facilitate reliable measurement and/or analysis. Sensing signal stimulation implementations of embodiments may, for example, reliably evoke sensing signals (e.g., ECAPs) using pinging-pulses in association with paresthesia-free stimulation (e.g., burst stimulation).

In addition to modification of waveforms and/or pulse patterns to facilitate sensing of ECAPs, certain embodiments conduct other operations to facilitate ECAP sensing. These operations may be performed for the waveform/pulse patterns discussed herein and may occur for paresthesia and non-paresthesia based therapies.

In some examples, ECAPs may occurs several milliseconds (ms) after the end of the stimulation delivery. In some implementations, a stimulation artifact can be a powerful voltage fluctuation during recording, and it may take some time to let the artifact to recover to the baseline after the stimulation ends. Often, stimulation recovery will overlap with the time window in which ECAPs signal appears, which makes the data sensing and analysis of ECAPs very difficult. Previous attempts to solve this problem use a pre-generated model to calculate the stimulation artifact recovery, and then subtract the model generated artifact recovery from the recorded signal to extract the ECAPs (Pilitsis J, et al., 2021). The disadvantage, however, is that the stimulation artifact will be different from day to day or from subject to subject, due to the movement of the recording electrodes in/on the body. Accordingly, the use of a pre-generated model to compensate for stimulation artifact in ECAP data can offer limited value.

In some embodiments, a neurostimulation system converts ECAP data in the time domain into some temporal-frequency domain signal. Then, temporal and frequency-based features are extracted to conduct signal denoising. After the feature extraction, the signal is converted back into time domain to obtain the relevant ECAP signal without noise or artifact. These processing operations are adaptive to each individual recording and there is no need to use pre-generated model. By employing processing operations in this manner, neurostimulation systems are capable of more accurately determining the effect(s) of neurostimulation and, thereby, improving patient therapy.

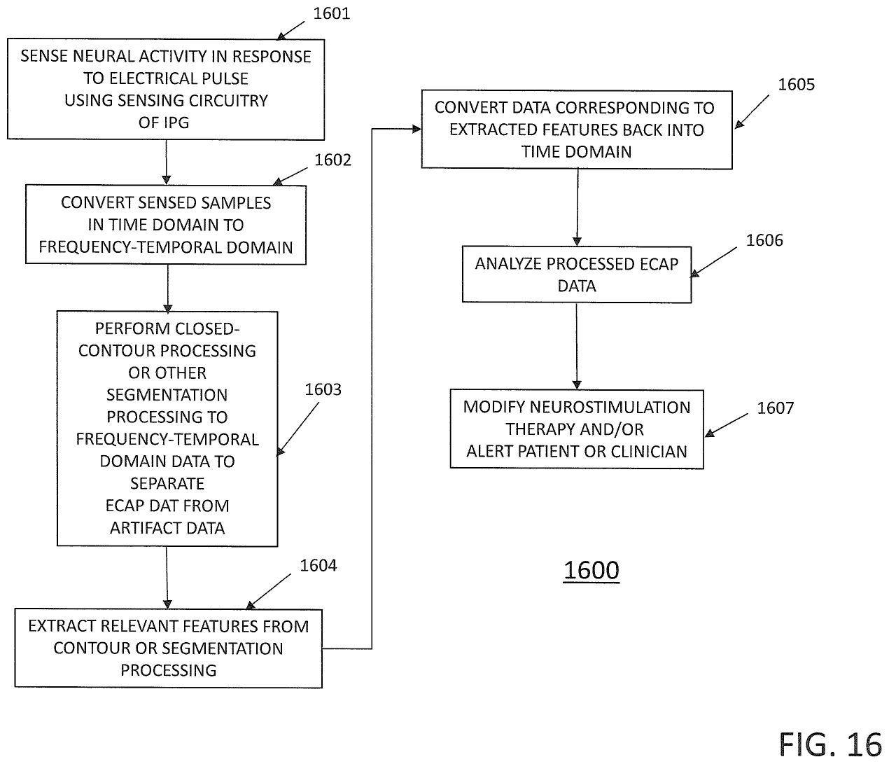

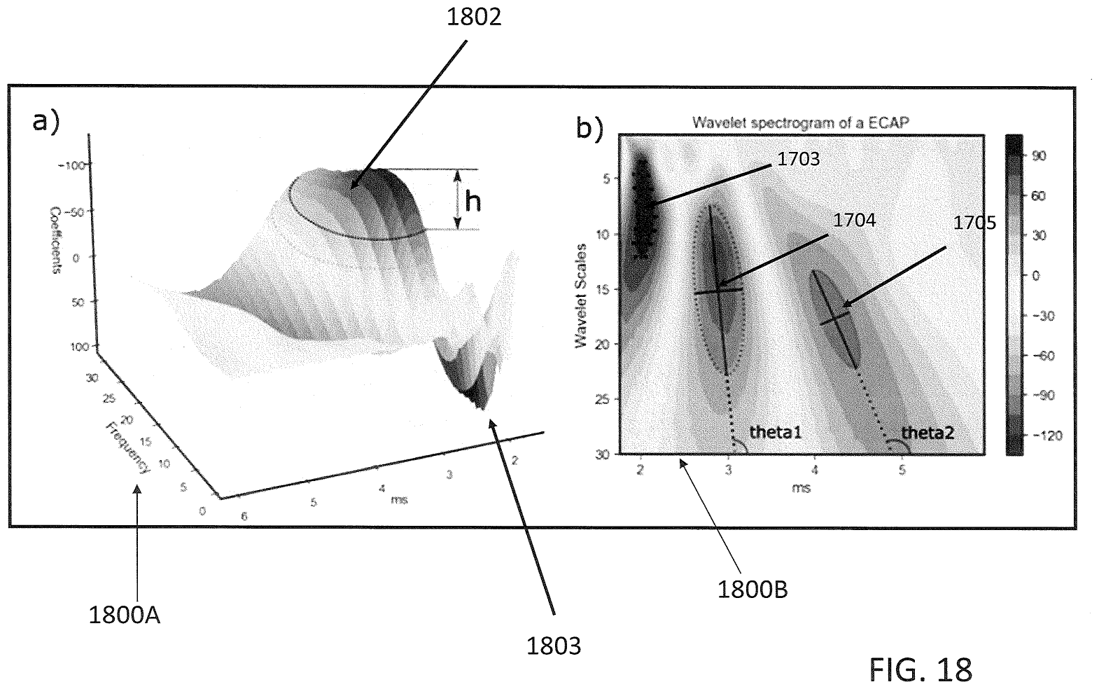

FIG. 16 depicts operations of a neurostimulation system for sensing and applying ECAP data according to some embodiments. In block 1601 of example process 1600, neural activity is sensed using suitable sensing circuitry of an implantable pulse generator (IPG). In some examples, the sensing circuitry may include sensing circuitry described in published literature and/or used in commercial neurostimulation devices or any later developed circuitry. In some examples, the sensing circuitry may include nerve action amplifier circuitry utilizing a hybridization of different types of transistor devices as will be set forth further below. For ECAP applications, the sensed neural activity is neural activity that is evoked by neurostimulation. Referring to FIG. 17, waveform 1701 represents sensed electrical activity corresponding to an electrical pulse and its evoked compound action potential. Due to the timing of the electrical pulse, recovery time, and the ECAP itself, the sensed waveform detected by the sensing circuitry may contain these various components. The sensed data may be truncated to exclude samples that include the stimulation artifact from the electrical pulse while retaining the artifact recovery component. The artifact recovery and the ECAP components in the sensed data of the truncated time window are processed as discussed herein to isolate the ECAP component for further operations of the neurostimulation system.

At block 1602 of FIG. 16, the digital samples of the sensed waveform in the time domain and within a relevant time window are converted to a suitable frequency-temporal domain. In some embodiments, the processing operations to transform to a suitable frequency-temporal domain include applying the wavelet transform.

Wavelets are mathematical functions that process data into different frequency components, and then study each component with a resolution matched to its scale. Wavelet processing has advantages over traditional Fourier methods in analyzing physical situations where the signal contains discontinuities and sharp spikes. In contrast to the varieties of Fourier analysis that appropriate observed data than the sines and cosines functions, wavelet analysis employs approximating functions that are contained neatly in finite domains. The windowed Fourier transform (WFT) is a known application of representing the nonperiodic signal in a frequency-temporal domain. With the WFT, the input signal f(t) (in this case the sensed data samples containing the stimulation artifact, the artifact recovery, and the ECAP) is segmented or divided into sections or time windows, and each section/time window is analyzed for its frequency content separately. If the signal has sharp transitions, the input data may be windowed so that the sections converge to zero at the endpoints. This windowing is accomplished via a weight function that places less emphasis near the interval's endpoints than in the middle. The effect of the window is to localize the signal in time.

The processing operations of transforming signal into frequency-temporal domain are not limited to wavelet transform but may also include methods for transforming signal into frequency or scales-like signal. Such methods might include Short Time Fourier Transform (STFT/DTFT), wavelet transform, Hilbert Transform, et al. according to other embodiments.

The processing of the time domain data into the frequency-temporal domain generates a two-dimensional array of data. The two dimensions are frequency and time. Each value in the two-dimensional array represents the signal power/amplitude at a given frequency at a given time. Graph 1702 represents a graph of the two-dimensional sensed data in the frequency-temporal domain according to some embodiments.

The two-dimensional array of data may be subjected to closed-contour analysis and/or other segmentation processing to extract ECAP related features in the data (block 1603 in FIG. 16). Specifically, in some example arrangements, the ECAP component(s) of the data may be determined as discrete phases at different temporal-frequency space locations. For example, graph 1702 includes closed contours 1703, 1704, and 1705, which each correspond to respective phases of the ECAP response of the patient to the electrical pulses. FIG. 18 depicts graph 1800A of the same data in the frequency-temporal domain in which a three-dimensional graph of the amplitude (e.g., as may be represented by a range of coefficients) versus time and frequency is provided by projecting into a graph format, wherein certain peaks/valleys (e.g., extrema 1802, 1803) may be identified. Upon ECAP feature identification, the longitudinal and time axis angle of each closed contour may be extracted (e.g., theta1 and theta2 as shown in the wavelet spectrogram 1800B of FIG. 18). The estimated time duration of each corresponding ECAP feature can be estimated from the identified closed contour features in the frequency-temporal domain data.

In some arrangements, 2D signal segmentation techniques may be employed to extract the closed-contours that contain relevant ECAP related data. Suitable signal segmentation techniques include thresholding, clustering, histogram-based filtering, edge-detection, regional property-based detection, or machine-learning and computer vision-based methods. Both, semantic and instance segmentation methods, may be employed to identify all contours and accurately classify them. Additionally, information about the contour shape is integrated to facilitate classification of regions as signals of interest versus noise (e.g., circularity, eccentricity, etc.).

After initial segmentation processing, additional/related features to may be extracted to help further screening, such as, e.g., features relating to ECAP signals or the targeted signal of interest. For example, extraction of amplitude-based features, the angle between the longitudinal axis and the time axis, the duration of the closed contour of the ECAPs like signal, etc. These features can be used to compare to or measure against known features or profiles of ECAP signals, and then further rule out those extracted features that are not ECAP-related or target signals of interest. At least some of these operations may be set forth as block 1604 of example process 1600 according to some embodiments herein.

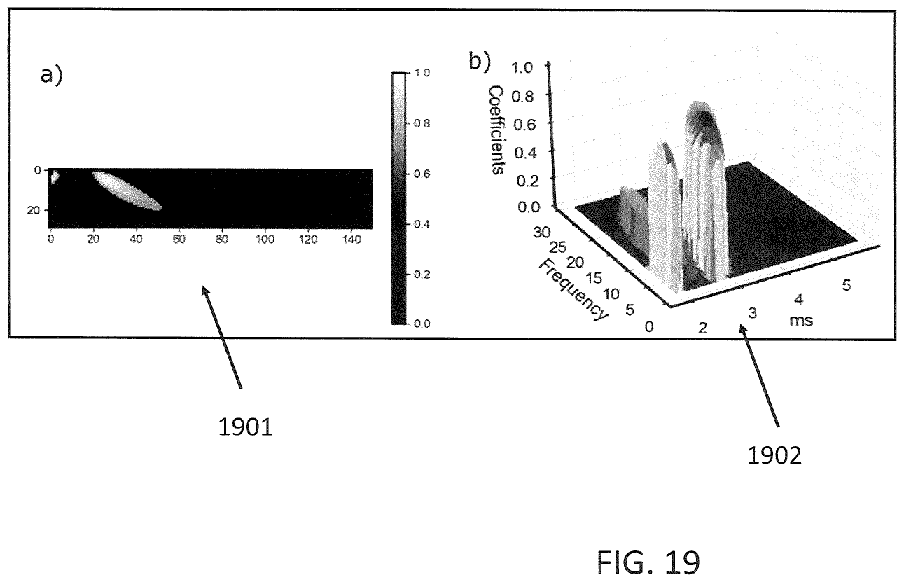

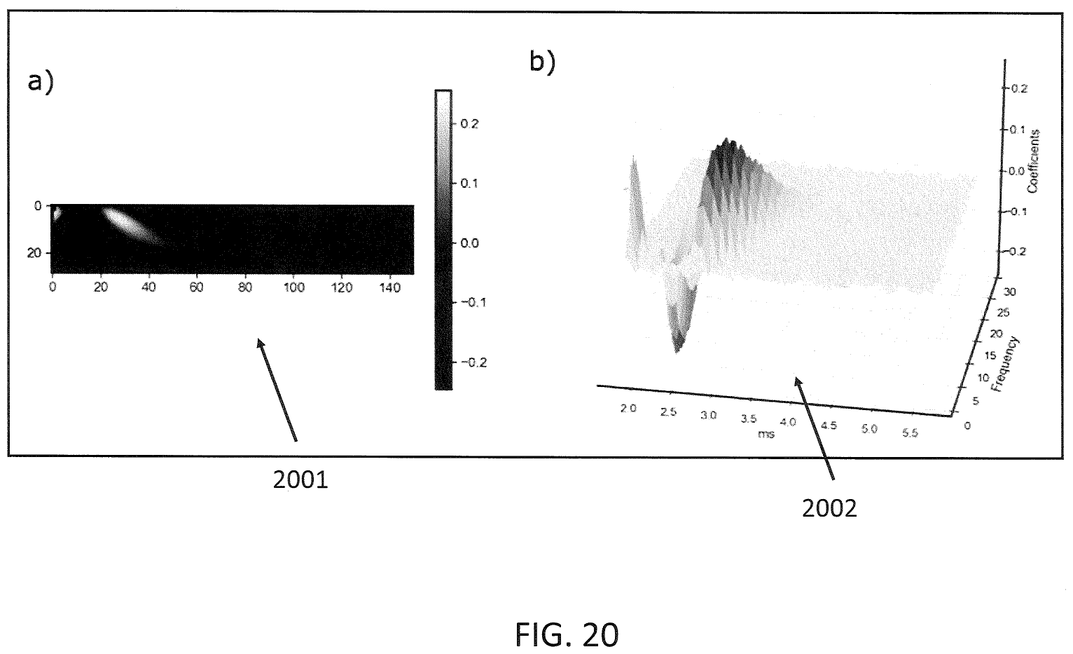

Referring to FIG. 19, graph 1901 depicts segmentation of the different phases of the ECAP response of the patient to the electrical pulse, wherein graph 1902 representing a 3-D graph of the frequency coefficients from the segmented data. After segmentation, processing of the remaining data may occur. The frequency coefficients for each segmented feature may be resettled or reset with the base of each feature beginning at or set to zero. Additionally, the remaining data points in the 2-D frequency-temporal domain that are not associated with one of the segmented ECAP features (i.e., zero data points) may be masked with Gaussian noise. In similar fashion, FIG. 20 shows another view of segmented ECAP phases and associated 3-D graph, respectively illustrated in graphs 2001 and 2002 thereof.

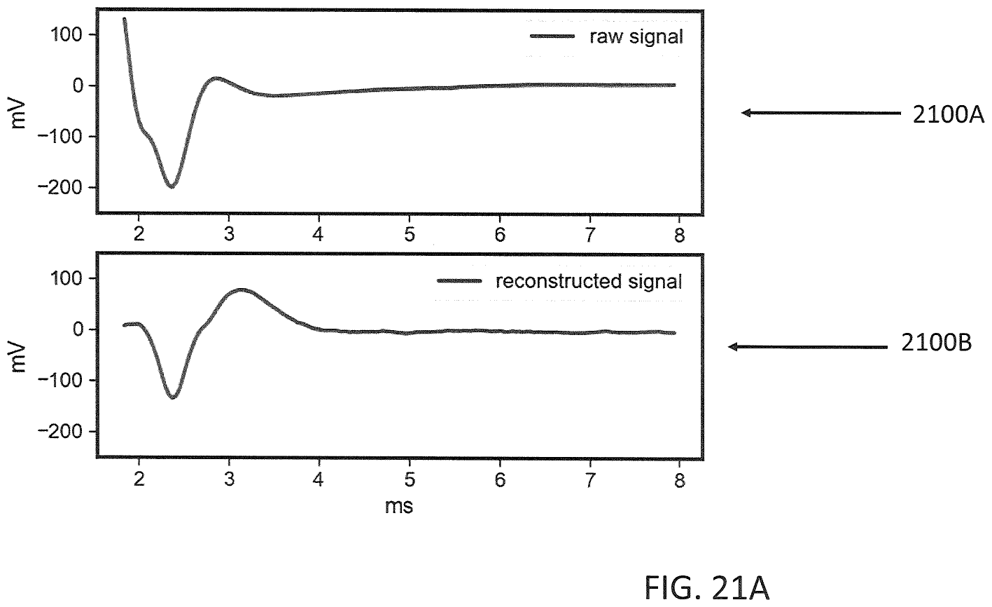

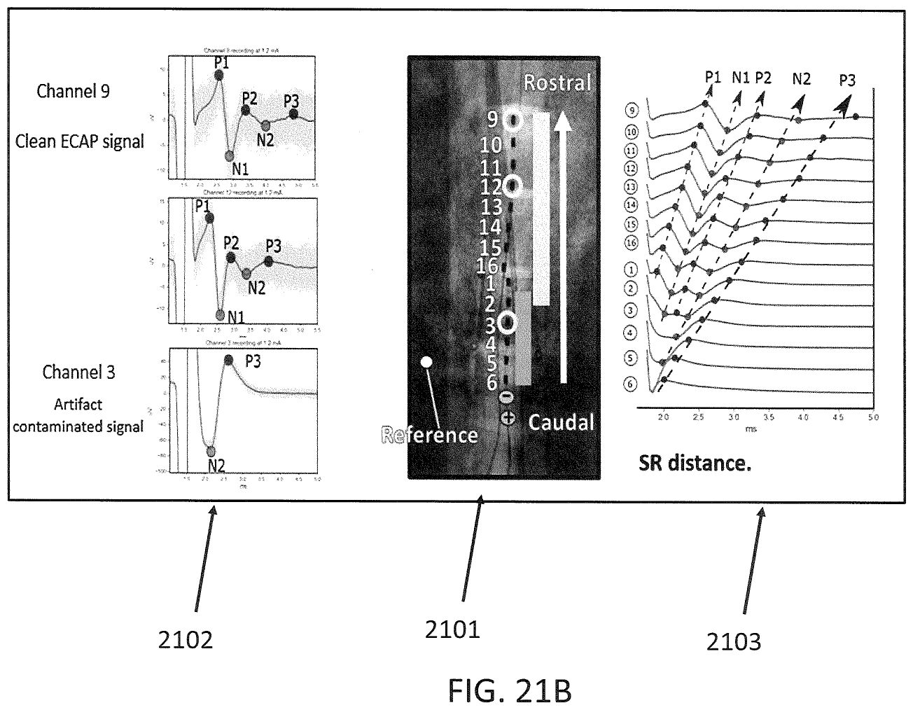

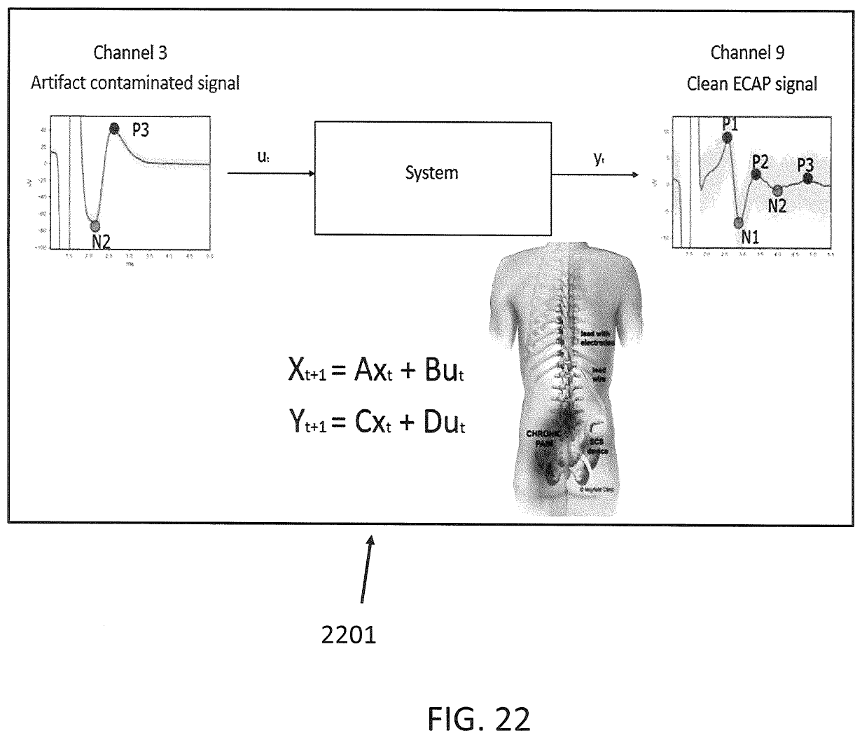

Referring back to FIG. 16, the 2-D array of data in the frequency-temporal domain corresponding to extracted ECAP features is converted back into time domain (block 1605). Any suitable transformation processing may be employed. The conversion may include application of a 2D kernel to smooth the signal and then transformation of the data into a one-dimensional time series. Alternatively, the conversion may include transformation of the data into a one-dimensional time series and then application of a 1 D kernel to smooth the signal. Referring to FIG. 21A, graph 2100A represents raw signal data obtain by sensing circuitry of an IPG and graph 2100B represents processing of the signal as discussed herein to generate the reconstructed signal that represents the segmented ECAP features.

Continuing to refer to FIG. 16, at block 1606, the reconstructed signal is analyzed (as discussed herein) to determine whether any appropriation action should be taken and, if so, the patient's neurostimulation therapy is modified and/or an alert is provided to the patient and/or the patient's clinician (block 1607).

The processing of ECAP data into a temporal-frequency domain may be used for any number of applications to assist neurostimulation therapies. In some embodiments, this analysis is conducted while a clinician implants one or more stimulation leads within a patient during a medical procedure. For example, the position of a stimulation lead is an important factor for successful neurostimulation therapy. If the stimulation is correctly positioned, the proper dorsal fibers of the spinal cord may be stimulated by electrical pulses applied through electrodes of the lead. Alternatively, the electrical pulses may stimulate unwanted neural tissue causing unwanted side effects (e.g., muscle stimulation or painful/uncomfortable sensory effects, etc. in the case of improper placement or positioning).

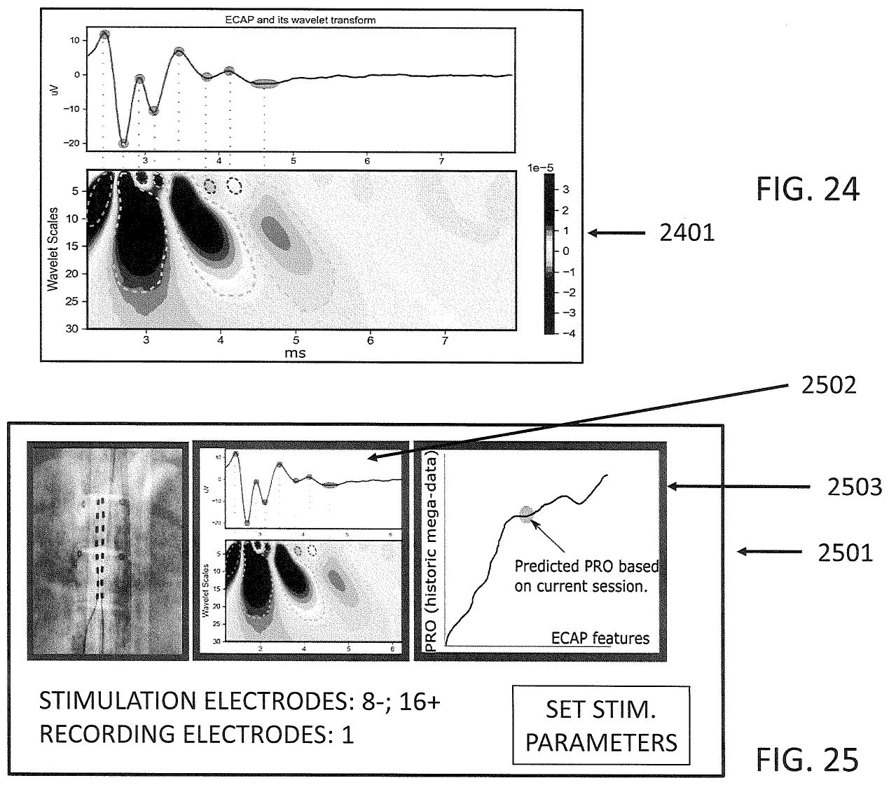

FIG. 24 depicts ECAP display 2401 according to some embodiments. As seen in display 2401, the ECAPs signal exhibits a complicated multiphasic morphology (as compared to the morphology of the ECAP signal seen in FIG. 17). ECAP display 2401 may be created by stimulating, measuring the patient response using a suitable electrode, and processing the signal as discussed herein (including the processing operations described with respect to FIG. 16). The display of the morphology shown in ECAP display 2401 may indicate unexpected activation in response to the stimulation (which might indicate the activation of muscle activity). When such a response is seen by the clinician during an implant procedure, the clinician may reposition one or more the stimulation leads before completing the implant procedure with the lead or leads in their final position.

Clinician user interface 2501 shown in FIG. 25 depicts display of ECAP data to assist an implant procedure according to some embodiments. Clinician user interface 2501 may be provided using a clinician programming device as discussed herein using a clinician “app” on the device. Clinician user interface 2501 may display a medical image of the patient with the position of the leads shown relative to the patient's anatomy. The medical image may be a virtual construction for the position or a direct image from medical imaging technology (e.g., a fluoroscopic imaging system). Clinician user interface 2501 may display the electrode(s) used for stimulation and recording operations. Clinician user interface 2501 may include a graphical user control element to switch to conventional programming screens (to set stimulation settings, e.g., amplitude, frequency, pulse width, etc.). Clinician user interface 2501 depicts ECAP data in the time domain and in the temporal/frequency domain as discussed herein as shown in ECAP display 2502. The implanting clinician may view ECAP display 2502 to ensure that the expected patient response to the stimulation is obtained at the implant position.

In some embodiments, ECAP measurement and objective analysis may be conducted real time during an implant procedure. The ECAP results may be displayed alongside the fluoroscopy image to provide guidance for physicians during the implantation process. In some embodiments, clinician user interface 2501 may also provide a predicted patient reported outcome (“PRO”) based on the current signals that the system is measuring during implantation process. The predicted may be conducted by a mathematical model that is trained based on historical offline data. During the implantation process, the newly measured ECAP signal may be feed into the model, and a predicted PRO outcome in PRO graph 2503 may be shown to inform physician about the effectiveness of the stimulation applied to the patient. This predicted PRO, together with ECAPs signal visualization, may be used to provide quantitative analysis real time during the implantation procedure in some example scenarios.

The PRO could be any patient reported outcome data from historical sessions from the same or other patients. The PRO data could consist of any type of patient reported outcome including, but not limited to, patient reported score, quality of life assessment score, paresthesia information from historical session or during the session reported by the patient, etc. The features used to train the model offline and conduct prediction during the actual implantation process could include ECAPs signal and its derived features, other types of physiological data recorded during a implantation session, such as somatosensory evoked response, ECG, EEG, etc. The features might also use patient reported results to feed in as an input to the model, based on how the predicted PRO is defined.

In other embodiments, samples of sensed data in response to electrical pulses applied to the patient may be subjected to other processing to remove stimulation artifact recovery features from ECAP features in the sensed data. FIG. 21B depicts a scheme for additional processing wherein ECAP data related to stimulation on caudal electrodes and sensed data using electrodes rostral to the stimulation electrodes may be exemplifed. As shown in lead configuration image 2101 depicted in FIG. 21B, two percutaneous leads are implanted in the epidural space of the patient. The electrodes of the two leads are roughly placed in a linear, sequential order. The percutaneous leads are placed such that the most distal electrode (“electrode 1”) of the first lead is immediately caudal to the most proximal electrode (“electrode 16”) of the second stimulation. Electrodes 7 and 8 (with polarities of “−” and “+”) are used to apply stimulation pulses from the IPG to dorsal fibers of the patient's spinal cord.

Because electrode 9 of the second stimulation lead has sufficient spatial separation from electrodes 7 and 8 of the first stimulation lead, it is possible to measure a clean ECAP signal. However, in channel 3 which is located very close from the stimulation channels, only artifact contaminated signal can be measured in certain circumstances. The stimulation artifact and artifact recovery features propagate more rapidly than the ECAP features. For sensing electrodes closer to the stimulation electrodes, there is overlap of the respective features in time. With electrodes with greater separation, there is sufficient temporal separation between features to allow ready identification of the separate features as represented in the timing graph 2103 of FIG. 21B.

As shown in sensed data graphs 2102 of FIG. 21B, the ECAP features including the multiple positive phases (P1, P2, and P3) and negative phases (N1 and N2) are identifiable from the sensed data using electrode (or channel) 9. However, for electrode (channel) 3, the sensed data is largely dominated by the artifact/artifact recovery features from the applied electrical pulse and, hence, the ECAP features are hidden in the sensed data from electrode 3.

As shown in FIG. 22, a patient system computational model may be created to facilitate recovery of ECAP features from a sensing electrode that is relatively close to one or more stimulation electrodes. As seen in computational model or relationships 2201 of FIG. 22, the signal of electrode/channel 3 (that may be contaminated by stimulation artifact/recovery features) is defined as the input of the patient system and the signal sensed by electrode/channel 9 (which has a clean ECAP signal) is defined as the output of the system. The patient system can be represented as a transfer function. To perform system identification and find a corresponding transfer function model, a state-space model is defined by matrices A, B, C and D. These matrices can be solved from the input and output signals by utilizing the Eigensystem realization algorithm (ERA) in an example implementation. Upon determination of these matrices A, B, C, and D, the system is characterized and, then, can be applied to subsequent data samples to obtain the ECAP features from a signal contaminated with stimulation pulse artifact/recovery features. That is, using the state-space model, it is possible to estimate output Yₜ (clean ECAP signal) from input uₜ (artifact contaminated signal).

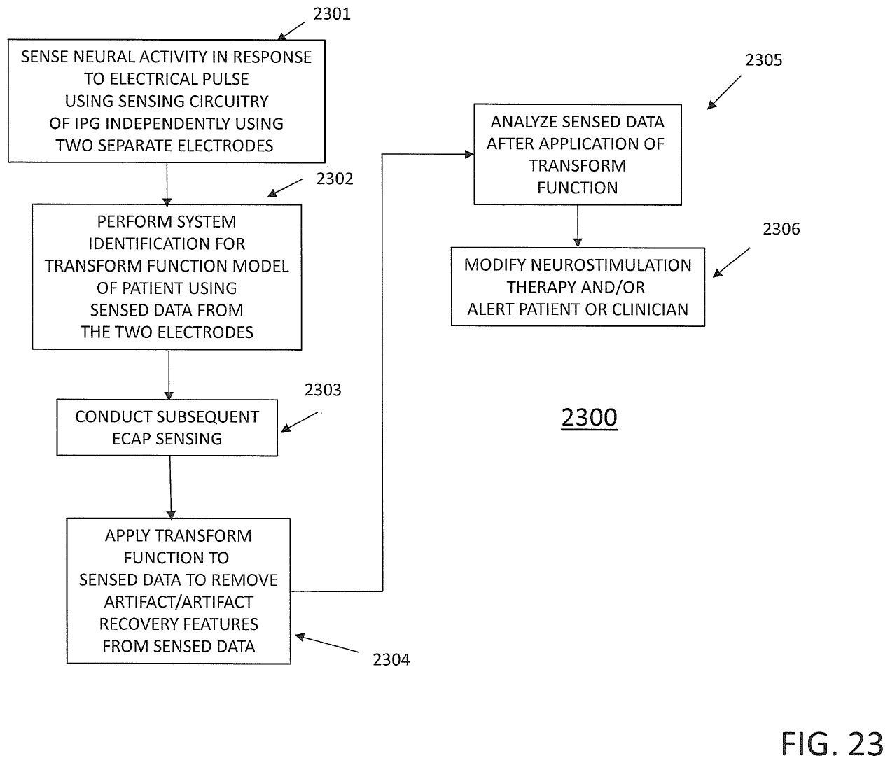

FIG. 23 depicts operations for conducting ECAP sensing operations for a neurostimulation system according to some representative embodiments. At block 2301 of example process 2300, neural activity in response to an electrical pulse is sensed using circuitry of IPG using two separate electrodes. At block 2302, system identification operations are performed using the sensed data from the two electrodes. The system identification operations determine a transform function that transforms that time series data sensed using the first electrode into the time series data sensed using the second electrodes. At block 2303, subsequent ECAP sensing operations may be performed in some optional arrangements. At block 2304, the transform function is applied to subsequently sensed data to remove artifact and/or artifact recovery features from the sensed data. At block 2305, the sensed data after application of the transform function is analyzed using suitable ECAP analysis operations. At block 2306, the neurostimulation therapy of the patient is modified and/or an alert is provided to the patient and/or the patient's clinician.

In some embodiments, ECAP analysis may be applied to detect migration of one or more stimulation leads after implantation into a patient. The detection of migration may detect relative movement of one stimulation relative to another stimulation lead. In other embodiments, detection of migration of a stimulation lead may detect migration of a stimulation lead relative to an anatomical structure of the patient. In addition, the migration (in either case) may be transient. For example, the position of the leads and/or their respective electrodes may change based on patient posture and some embodiments detect such changes. The detected change may be used to infer patient posture, position, activity, and/or the like and stimulation may be modified as appropriate.

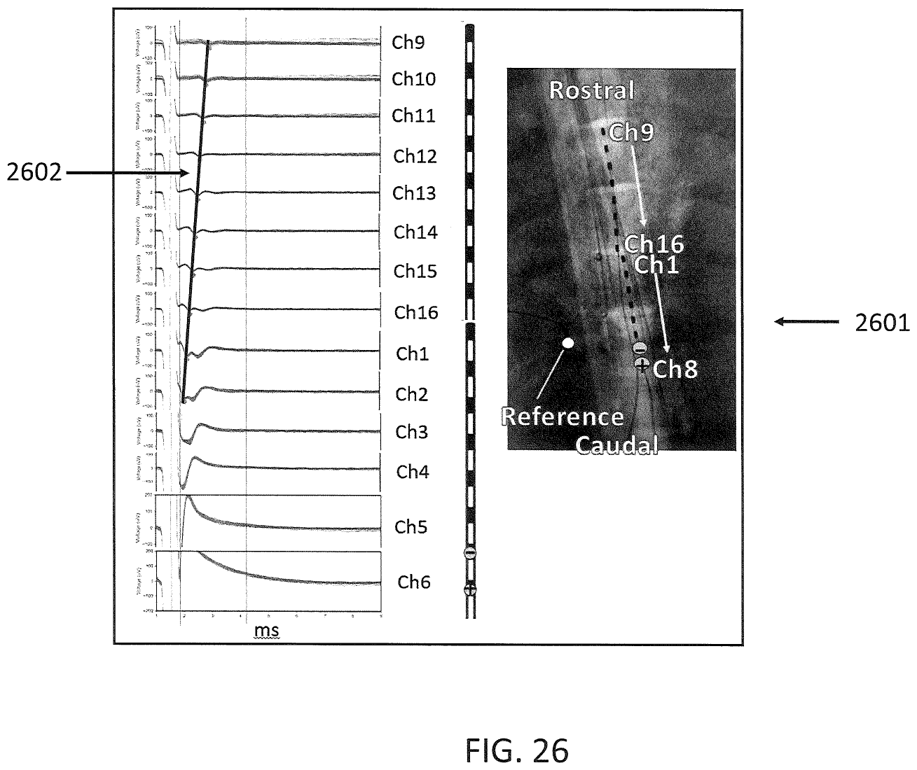

FIG. 26 depicts the propagation of ECAP from stimulation electrodes occurring rostrally along respective recording electrodes along the depicted stimulation lead. As shown in FIG. 26, electrodes of channels 5 and 6 (shown as Ch5 and Ch6) as used as a bipolar pair (one negative and one positive electrode) for application of a stimulation pulse. The electrical pulse causes an ECAP and the ECAP propagates through the neural tissue. In this case, Ch1-Ch4 of the same stimulation lead and Ch9-Ch16 of a second stimulation lead positioned substantially parallel and rostrally to the first stimulation lead as exemplified in image 2601, which can be used to measure the propagating ECAP. The detected waveform including the ECAP is shown in time adjacent to each electrode or channel. Line 2602 shows the beginning of the ECAP feature in time as detected by each respective electrode or channel. Near the stimulation site (more caudally), the evoked response has shorter latency from the stimulation artifact, while away from the stimulation site (more rostrally), the evoked response has longer latency from the stimulation artifact.

In some embodiments, the ECAP signal can be used to detect the relative position of multiple leads (e.g., two stimulation leads commonly implanted). Stimulation is initiated by one of the implanted leads and ECAP neural recordings are recorded on the other lead. Across multiple contacts of the leads, ECAPs signals will present different phase shift or latency depends on the location (as shown in FIG. 26).

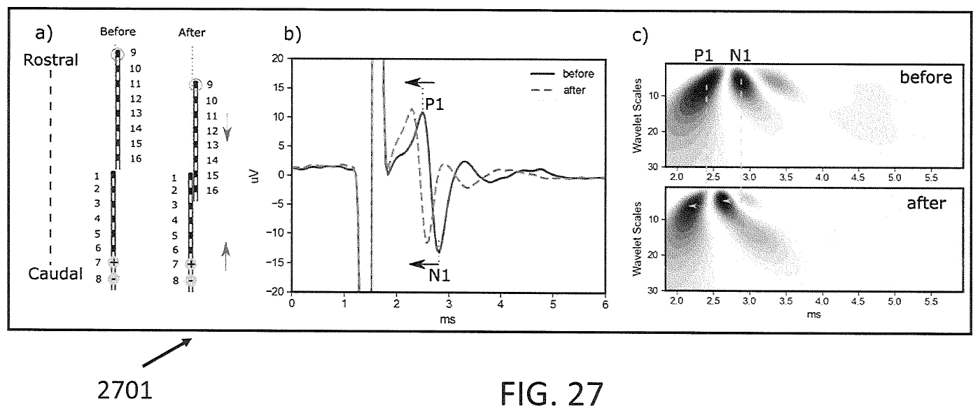

The phase shift or latency change could be viewed in both time domain in regular time series data, or it can be viewed in the transformed domain (as shown in graph 2701 of FIG. 27). The example shows the transformed domain as the scalogram of using wavelet transform applied on the original time series recording. The methodology used in such transform could also include any other similar signal processing methods, such as Fourier Transform based methods (FFT, DTFT), Hilbert Transform based methods (HHT), which can generate any spatial-temporal, frequency/scale-temporal signal map to visualize the latency change.

In one example, the latency of the ECAPs recording (as shown in FIG. 27) indicates the distance between the two leads. In this manner, a diagnostic can be run when the subject is known to be in a particular position (e.g., lying down when sleeping) and the latency of the ECAP neural signal following the stimulation pulse may be used to determine the relative position of the two leads in the rostro-caudal direction. For example, given any contact on the recording lead, if with decreasing latency at different time of measurements, the rostral lead is moving more caudally or the caudal lead is moving more rostrally. Similarly, if the recorded ECAP signal is with increasing latency at different measurement time, it is likely that the rostral lead is moving rostrally, or the caudal lead is moving more caudally.

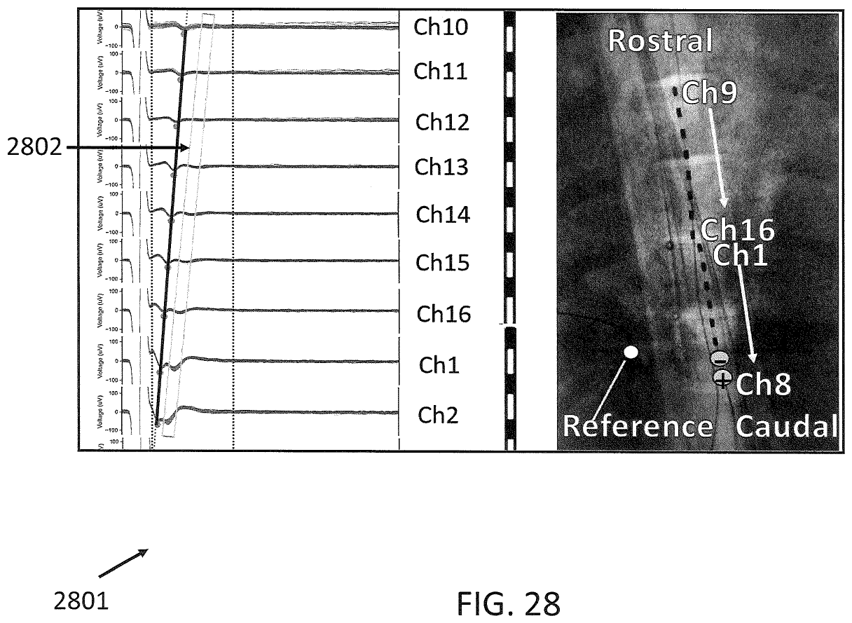

In another embodiment involving multiple implanted leads, the emergence and/or disappearance of a secondary phase of the ECAPs may happen as a recording contact is moving towards the stimulation location (as highlighted by movement direction 2802 in graph 2801 of FIG. 28). In the case of emergence of new phase of ECAPs signal for a given contact, it indicates the rostral recording lead might be moving more caudally, or the caudal stimulation lead is moving rostrally. In the case of the disappearance of the secondary phase of the ECAPs signal, it indicates that the rostral recording lead might move more rostrally, or the caudal stimulation lead might move more caudally.

Referring to FIG. 29, the disappearance of a phase (P1) of ECAPs signal is shown in graph 2901. When two leads are moving towards to each other, either recording lead is moving more rostrally, or stimulation lead is moving more caudally. The first positive peak (P1) from the same recording channel is completely disappeared after the lead has moved. This may be observed in both time series (subplot b) and scalogram (subplot c).

As shown in the respective graphs, the timing of the phases (arrival, disappearance, etc.) can be identified in the time domain and/or in the temporal/frequency domain. The various techniques discussed herein may be applied to record data for the ECAP analysis.

The timing analysis discussed herein is not limited to application on the ECAP signal that is recorded after the stimulation delivery. In the case of no ECAPs being triggered, the waveform or its transformation of the artifactual recording could also be used to conduct the time domain or transform domain analysis to track if there is any signal latency or phase changes.

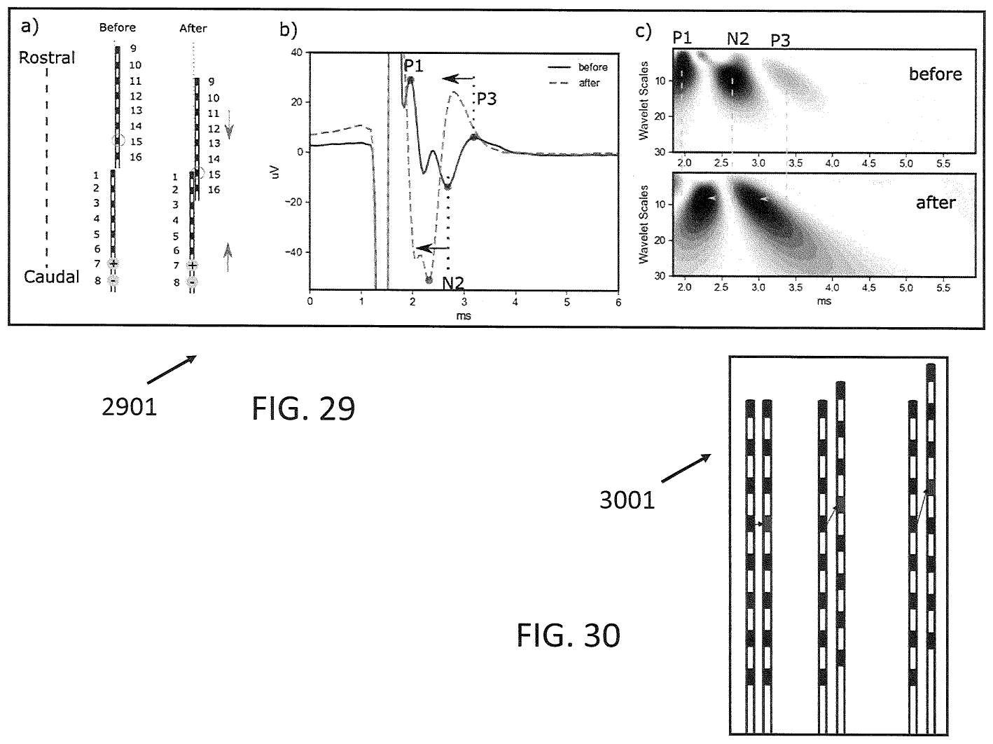

In other embodiments of multiple implanted leads, at least one electrode contact from each lead is used to generate ECAPs. The ECAP is generated by the activation of the dorsal column axons and the axonal activation is maximized when the electric field is aligned in the axonal direction (as shown in lead arrangements 3001 of FIG. 30). With lead migration, the angle between the electric field and the axonal direction changes, which may result in a change in the percentage of the activated axonal population and the ECAP signal amplitude and morphology. Different stimulation amplitudes, configuration and recording configuration may be used and the morphology profile of ECAP characteristics including the peak-to-peak value, the shape of ECAPs and the latency, etc. may be assessed. Different configurations of differential recording can be also used in some arrangements. With migration, the morphology of ECAP including the peak-to-peak value shape and latency may be also affected. Combinations of different stimulation electrode pairs and differential recording pairs may be implemented and the profiles of ECAP signals corresponding thereto may be recorded, wherein a migration may alter the ECAP profiles. In addition to the change of the angle between the electric field and the axonal direction, the change of the surrounding structure of the stimulation and the recording lead may affect the profile of the ECAPs with lead migration. The ECAP threshold amplitude may change when the stimulation electrode contacts are under bony vertebrae compared to inter-vertebral space. In addition to the change of the angle between the electric field and the axonal direction, the change of the distance between anode and cathode may affect the electric field strength resulting in ECAP profile changes.

Whereas some example embodiments set forth above have been particularly described in reference to an implantable device, it should be understood that biosignal activity (e.g., ECAPs) may also be monitored in NIMI devices or partially-implanted devices for purposes of adjusting therapy settings in some implementations. Further, more than one pulse generating circuit (also referred to herein as a stimulation engine) may be provided as part of an IMD/NIMI device for facilitating highly controllable therapeutic waveforms in conjunction with biosignal-based adjustability or therapy modulation, wherein an individual stimulation engine (SE) may be (re)configured by varying a number of parameters, such as selection from a plurality of electrodes disposed on one or more stimulation leads, variation of the stimulating currents, and control over the duration, rates, and pulse sequencies associated with current delivery, etc. Regardless of whether a single SE or a multi-SE arrangement is deployed, a switching matrix may be provided in some example embodiments wherein one or more electrodes may be selectively and switchably coupled to a suitable sensing circuit for sensing one or more biosignals in some embodiments as will be set forth in detail further below. Depending on implementation, some of the electrodes may be configured as sensing electrodes, stimulation electrodes, or both (that is, electrodes that may be configured for stimulating a target tissue in a stimulation event and then reconfigured subsequently as sensing electrodes after or within a predetermined time period in a sensing event for sensing the biosignal activity). Irrespective of a particular implementation, it is desirable that nerve activity is monitored, sensed and/or recorded using a circuit that is optimized not only for form factor but also with respect to signal noise as well as power requirements because of the operating constraints of an IMD/NIMI device. Furthermore, where the electrodes are selectably coupled to the sensing circuit, it is desirable that additional switching functionality is provided that is optimized for quickly discharging and/or recharging respective capacitive loadings of the electrical connections in order that appropriate timing relationships between stimulation and sensing events are maintained.

In some embodiments, IPG 12 or another suitable IMD or NIMI device may therefore be adapted to include sensing circuitry for amplifying neural or other biosignal activity that may be sensed by one or more electrodes, wherein a switch matrix may be used for selectively coupling different electrodes of one or more stimulation leads to the sensing circuitry. In an example arrangement, the sensing circuitry may be implemented as a combination or a hybridization of integrated circuits (ICs) or transistors that are based on different semiconductor technologies such as bipolar junction transistors (BJTs), complementary metal-oxide-semiconductor (CMOS) field effect transistors (FETs), etc. wherein different portions of the sensing circuitry or assembly may be optimized for different performance parameters (e.g., input noise, power consumption or operating current, etc.), while achieving the overall design objectives with respect to a particular IPG/IMD/NIMI device implementation. In general, example embodiments herein may be configured to provide a sensing amplifier circuit that is compact, generates low equivalent input noise, and consumes currents (hence power) commensurate with a battery-powered medical device having a finite operating life span, wherein a synergistic combination of a noise-optimized input stage comprising one or more BJTs is electrically coupled to a power-optimized output stage comprising one or more CMOS FETs as will be set forth further below.

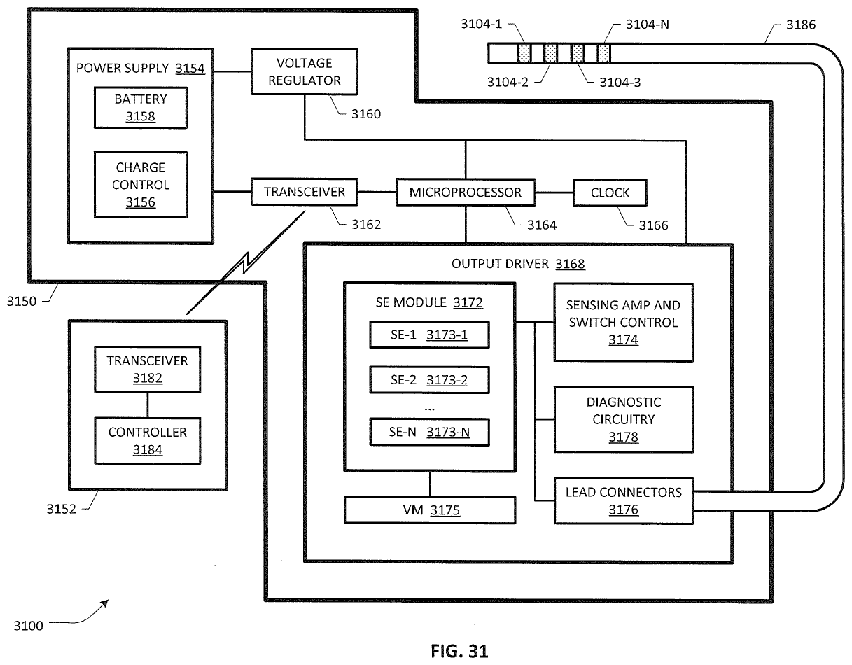

Turning to FIG. 31, depicted therein is a block diagram of a biostimulation system having one or more stimulation engines or sets of stimulation circuitry and associated sensing amplifier circuitry for sensing biosignals according to an example embodiment. Stimulation system 3100 is configured to include a generator portion, shown as IPG 3150, providing a stimulation or energy source, a stimulation portion, shown as lead system 3186 for application of the stimulus pulse(s) similar to the arrangements set forth in FIGS. 1A-1C and 2 described above, and an optional external controller, shown as programmer/controller 3152, to program and/or control IPG 3150 via a wired/wireless communications link, similar to the external device 46 described previously. IPG 3150 may be implanted within the body of a human or animal patient (not shown) for providing electrical stimulation from IPG 3150 to a selected area of the body via lead 3186 under control of external programmer/controller 3152. It should be appreciated that although lead 3186 is illustrated to provide a stimulation portion configured to provide stimulation remotely with respect to the generator portion 3150 of stimulation system 3100, a lead as described herein is intended to encompass a variety of stimulation/sensing configurations including, e.g., a microstimulator electrode disposed adjacent to a generator portion. Further, although example lead system 3186 is exemplified as a single implantable lead coupled to lead connectors 3176, the teachings herein are not necessarily limited thereto. An example embodiment of the present invention may, therefore, involve a lead system comprising two or more implantable leads, with each lead having a respective plurality of electrodes, wherein different combinations of electrodes/leads may be grouped into one or more channels in a stimulation therapy system including biosignal sensing. In some configurations, stimulation current pulses according to different therapies may be applied by one or more respective stimulation engines or sets of stimulation circuitry to different portions of electrodes based on a particular channel selection scheme regardless of whether one or more leads and/or one or more sets of electrodes are selected for stimulation, whereby one or more different sets of evoked biosignals may be monitored using a sensing amplifier (SA) arrangement of the present disclosure.

IPG 3150 may be configured to include a voltage regulator 3160, power supply 3154, transceiver 3162, microcontroller (or microprocessor) 3164, clock 3166, and output driver circuitry 3168 comprising a stimulation engine module 172 having one or more stimulation engines (SEs) 3173-1 to 3173-N, current regulation circuitry, switchable connectivity to a voltage multiplier (e.g., VM 3175) as well as discharge switching circuitry operable with a sensing amplifier circuitry 3174, which will be described in further detail below. Additionally, suitable diagnostic circuitry 3178 may also be provided as part of output driver 3168 in some embodiments. Power supply 3154 provides a source of power, such as from battery 3158 (which may comprise a non-rechargeable battery, e.g., single use battery, a rechargeable battery, a capacitor, and/or like power sources), to other components of IPG 3150, as may be regulated by voltage regulator 3160 including and/or facilitating digitally programmable analog voltage generation. Charge control 3156 of IPG 3150 is operative to provide recharging management with respect to battery 3158 in some optional, additional and/or alternative embodiments. Transceiver 3162 of an example embodiment of IPG 3150 is operative to provide data/control communication between microprocessor 3164 and a controller 3184 of external programmer/controller 3152, via transceiver 3182 provided therewith. Transceiver 3162 of an example embodiment, in addition to or in the alternative to providing data/control communication, may provide a conduit for delivering energy to power supply 3158 via RF or inductive recharging in some examples.

Microprocessor/controller 3164 provides overall control with respect to the operation of IPG 3150, such as in accordance with a program stored therein or provided thereto by external programmer/controller 3152, in addition to controlling tuning operations relative to configuring, setting and/or selecting the operating parameters of sensing amplifier circuitry 3174 in some embodiments, as will be set forth further below. One or more SEs 3173-1 to 3173-N may be configured to generate and deliver stimulation therapies having suitable pulse characteristics to selected sets or portions of electrodes 3104-1 to 3104-N under control of microcontroller 3164. In general operation, for example, different SEs 3173-1 to 3173-N of SE module 3172 may be controlled to output optimized stimulation therapies simultaneously or otherwise to different sets of electrodes selected under programmatic control. By way of illustration, a stimulation therapy may comprise delivering a constant current pulse of a desired magnitude/amplitude, duration, phase, and frequency to a tissue load present with respect to particular ones/sets of electrodes 3104-1 to 3104-N, which may be represented as respective lumped-element electrode/tissue interface (ETI) loads. Additionally, select ones of electrodes may be coupled to the sensing amplifier circuitry 3174 for sensing one or more biosignals developed at the tissue interface (e.g., ECAP signals as described above). Clock 3166 may be configured to provide system timing information, such as may be used by microcontroller 3164 in controlling system operation, as well as for different portions of SE module 3172 and/or VM 3175 in generating desired voltages, controlling switchable connectivity between the electrodes and one or more SEs 3173-1 to 3173-N and/or the sensing amplifier circuitry 3174 according to some embodiments.

In one example embodiment of IPG 3150, voltage regulator 3160 may be configured to accept a reference voltage V_{REF}, which may be prone to variation in magnitude, and provide an output voltage VOUT having a selected, relatively constant magnitude. For example, V_{REF} may be provided by battery 3158 which may have a relatively high voltage when initially charged or put into service and the voltage may drop over the life or charge cycle of the battery. However, circuitry of IPG 3150 may malfunction if a voltage applied thereto is not within particular limits, and the high and low voltage extremes associated with battery 3158 may be outside of these limits in some instances. Accordingly, voltage regulator 3160 may be configured to provide a regulated supply VOUT within a range acceptable to the various circuit portions of IPG 3150, including output driver circuitry 3168 and associated the sensing amplifier circuitry 3174, wherein the regulated supply VOUT and/or its derivative voltages may be operable as one or more local voltage references with respect to effectuating sensing operations for purposes of an example embodiment of the present disclosure.

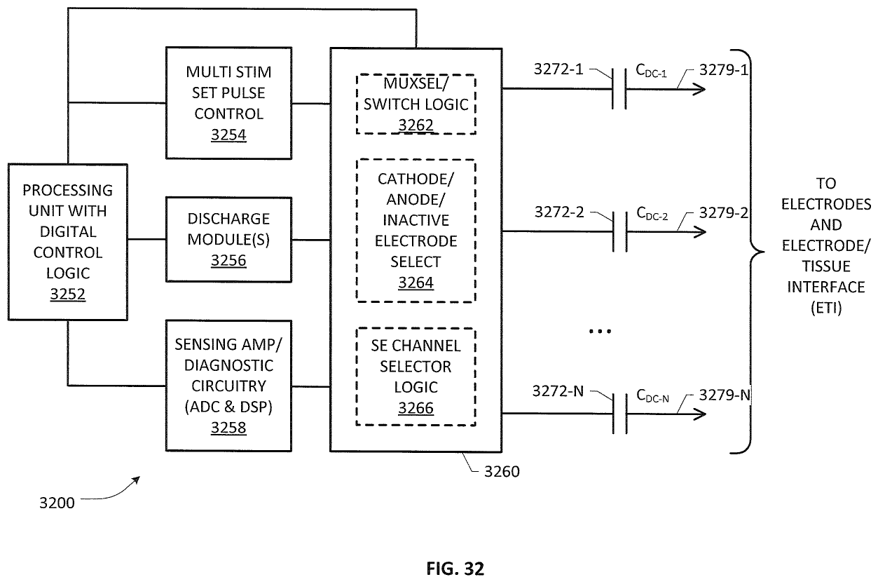

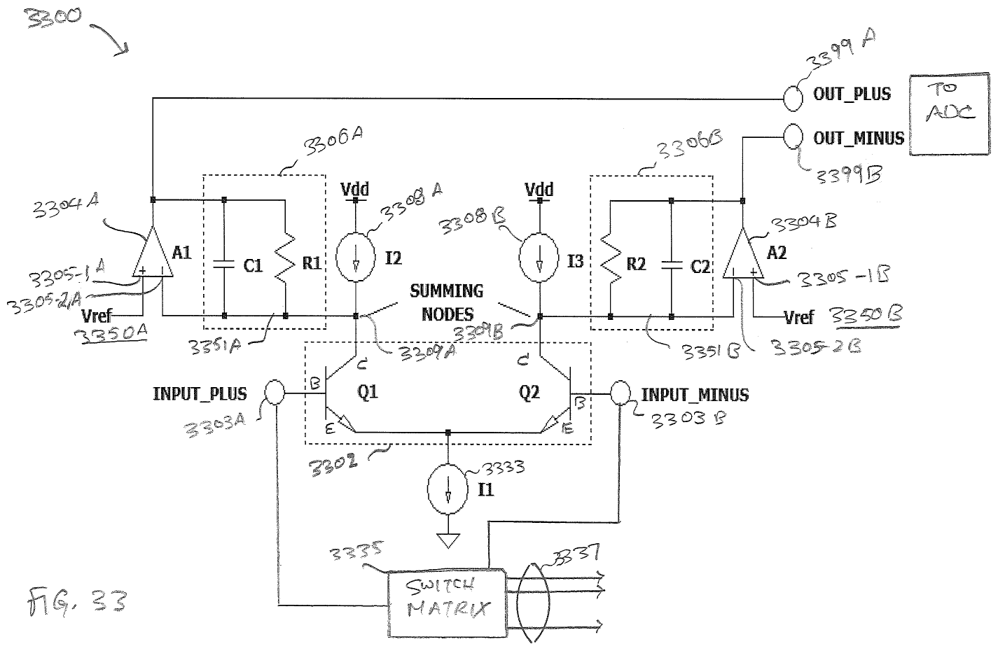

FIG. 32 depicts a block diagram of a pulse generator portion having pulse set control and lead electrode selection control wherein a sensing amplifier circuit may be switchably coupled to a portion of electrodes for sensing a biosignal from a tissue interface according to some embodiments. Skilled artisans will recognize upon reference hereto that various functionalities associated with example blocks shown as part of the pulse generator portion 3200 may be distributed and/or integrated among one or more blocks, subsystems and/or modules described hereinabove with respect to the IPG embodiments illustrated in FIGS. 2 and 31. Consistent with the description set forth above, a processing unit 3252 having or associated with suitable digital control logic is operatively coupled to SE pulse control module 3254, one or more discharge modules 3256 and sensing amplifier circuitry and diagnostic circuitry 3258 for facilitating various functionalities including but not limited to voltage measurements based on monitored biosignals, active discharge cycling, electrode selection and configuration, SE selection, etc. as well as generating appropriate control signals for adjusting one or more pulse set control parameters under appropriate programmatic/diagnostics control. In some arrangements, the diagnostic circuitry 3258 may also include circuitry configured to perform one or more sensed signal analyses and techniques set forth hereinabove. An input/output (I/O) interface block 3260 is operatively coupled to a plurality of lead connectors 3279-1 to 3279-N interfaced with respective electrodes, which interfaces may be modeled as suitable lumped-element ETI circuit representations, wherein the lead connectors and associated electrodes may be configured as one or more leads, each having a respective plurality of electrodes. Regardless of the number of leads, a lead connector 3279-1 to 3279-N may be provided with a DC blocking stimulation capacitor (CDC) for facilitating direct current flow blocking functionality with respect to the corresponding electrode that may be configured to operate as a stimulation node. Although some of the electrodes may also be configured to operate as sensing nodes in addition to providing stimulation (e.g., having an AC-coupling sense capacitor (C_{SENSE}) in addition to the DC blocking stimulation capacitor), such arrangements are not shown herein without loss of generality. By way of illustration, DC blocking stimulation capacitor CDC-1 3272-1 is coupled to lead connector 3279-1. Likewise, remaining lead connectors 3279-N may be provided with respective CDC-N 3272-N to facilitate DC blocking with respect to each corresponding lead electrode thereof.

Interface block 3260 may include appropriate multiplexing and selection/switching logic circuitry 3262 and anode/cathode/inactive electrode selection circuitry 3264 for measurement and sensing/diagnostics purposes wherein different electrodes of an electrode grouping of the lead system may be selectively configured for stimulation (e.g., anodic or cathodic stimulation), sensing, or designating unused/inactive states, etc., with appropriate electrical connections being made within an IPG device accordingly relative to the various components therein. In some embodiments, portions of diagnostic circuitry 3258 may comprise suitable analog-to-digital converter (ADC) circuitry configured for digital voltage measurement and associated signal processing using known or heretofore unknown voltage measurement techniques. As such, voltage measurement circuitry can be external and/or internal, on-board or off-board, and/or may be coupled to other measurement devices or instrumentation configured to perform some of the methods and processes set forth hereinabove depending on the particular biostimulation deployment, including reconstruction of biosignals, ECAP feature isolation and recovery, etc.

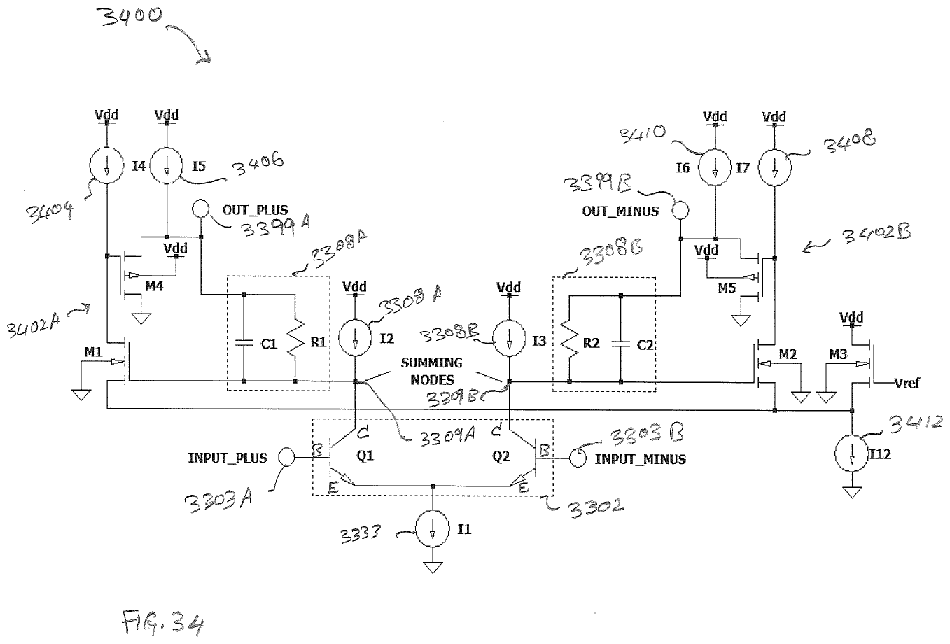

FIG. 33 depicts a schematic diagram of a sensing amplifier (SA) circuit or assembly that may be provided as part of an IPG/IMD/NIMI device for sensing biosignals according to an example embodiment. In one arrangement, SA circuit assembly 3330 may be implemented as a combination of a noise-optimized input stage 3302 coupled to one or more power-optimized output stages 3304A, 3304B. In one arrangement, the input stage 3302 and the output stages 3304A/B may be disposed on different semiconductor dies, and may preferably comprise active devices, e.g., transistors, that are fabricated using different technologies. In an example implementation, input stage 3302 may include one or more BJTs, e.g., Q1 and Q2, and the output stage 3304A/3304B may comprise respective amplifiers, e.g., A1 and A2, integrated into a CMOS-based application-specific integrated circuit (ASIC), whose operational functionalities will be set forth in detail further below. In an example configuration, noise-optimized Q1 and Q2 transistors of the input stage 3302 may be connected as follows: respective BJT emitters (E) are coupled to a common current source I1 3333, respective base terminals (B) are coupled to corresponding input nodes INPUT_PLUS 3303A and INPUT_MINUS 3303B, and respective collectors (C) are coupled to corresponding summing nodes 3309A and 3309B. In one arrangement, summing nodes 3309A/3309B may each be coupled to a respective trimmable current source I2 3308A and I3 3308B. Further, each summing node 3308A, 3308B may be coupled to drive a respective low-pass filter circuit 3306A/3306B having configurable gain factors, wherein the filter circuits 3306A/3306B are coupled to respective inverting input nodes 3305-2A and 3305-2B of amplifiers 3304A and 3304B. In one configuration, noninverting input nodes 3305-1A and 3305-11B of amplifiers 3304A, 3305B are driven by respective reference voltages (V_{REF}) 3350A, 3350B, which may be the same in some example arrangements. Each amplifier 3304A, 3304B is operable as a respective low-power tunable gain circuit forming an output leg configured to drive a corresponding output, OUTPUT_PLUS 3399A and OUTPUT_MINUS 3399B, which may be coupled to differential input nodes of an ADC circuit of suitable diagnostic circuitry of an IPG/IMD/NIMI device.

With respect to the input side of SA circuitry 3300, INPUT_PLUS 3303A and INPUT_MINUS 3303B of the input stage 3302 may be coupled to a switch matrix 3335 in an example arrangement, wherein the switch matrix 3335 may be configured to switchably couple INPUT_PLUS 3303A and INPUT_MINUS 3303B to a select pair of electrodes via corresponding lead connecters 3337.

In one implementation, current source (11) 3333 is operative to supply a constant current through the emitters (E) of BJT devices Q1 and Q2, which may be configured to divert the current in a differential fashion proportional to voltage difference between INPUT_PLUS 3303A and INPUT_MINUS 3303B. As noted above, the collector (C) of each BJT device is connected to respective summing node 3309A/3309B, driven by corresponding current sources I2 3308A and I3 3308B. Each summing node 3309A, 3309B, feeds into respective amplifiers 3304A and 3304B (at respective inverting inputs 3305-2A, 3305-2B via corresponding feedback paths 3351A, 3351B) such that either amplifier acts to maintain V_{REF} potential 3350A, 3350B at the respective summing node 3309A, 3309B via correction currents flowing through a first RC arrangement of filter 3306A (e.g., R1 and C1) and through a second RC arrangement of filter 3306B (e.g., R2 and C2). In one implementation, the RC arrangements of filter circuits 3306A/3306B may be disposed external to the CMOS ASIC chip comprising A1 and A2 circuitry such that RC value trimming functionality is eliminated from the CMOS ASIC chip. Consequently, die area utilization of the CMOS ASIC chip may be minimized in an example embodiment. In an alternative embodiment, however, the RC components of filters 3306A/3306B may be integrated within the CMOS ASIC die depending on the implementation. Regardless of how the RC components are configured, it should be appreciated that the corrective current passing through R1/R2 resistors and C1/C2 capacitors is operative to establish each output leg's voltage gain and frequency response in a configurable manner, wherein respective amplifier outputs may be tunable to meet the performance requirements of different deployment scenarios. In one arrangement, the tunable outputs may be presented as differential outputs, OUT_PLUS 3399A and OUT_MINUS 3309B, as noted hereinabove.

In an example implementation, accordingly, I2 3308A and I3 3308B may be advantageously configured as trimmable devices operative to serve two functions. For example, each source may be configured to supply constant currents to respective summing nodes, thereby reducing the steady-state voltages developed across R1 and R2 such that the voltage fluctuation (AV) between OUT_PLUS and OUT_MINUS nodes is increased and/or maximized. Further, I2 and I3 may be adjusted to compensate for production variation among the BJTs of the input stage 3302, e.g., base emitter voltage (V_{BE}) variation between Q1 and Q2.

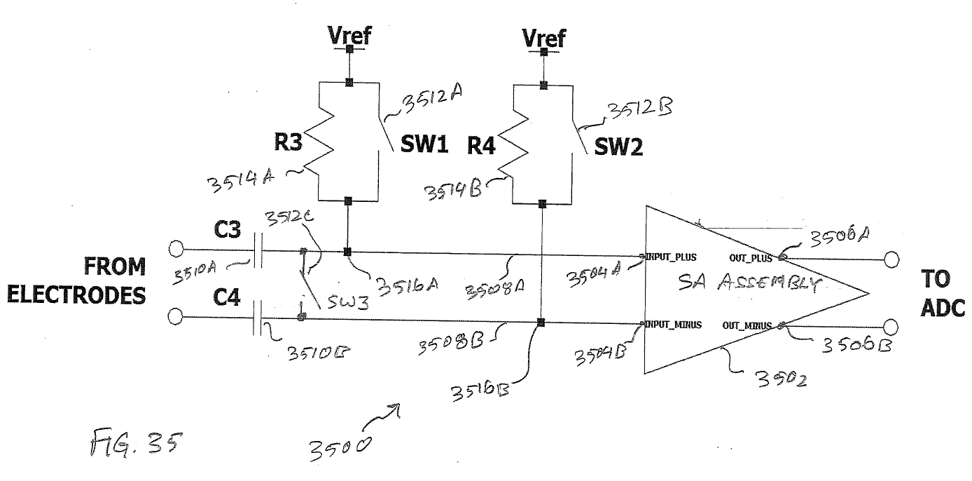

FIG. 34 depicts a circuit schematic diagram of an SA assembly according to an embodiment wherein a particular implementation of a CMOS-based output stage is exemplified with additional detail. As before, noise-optimized BJT-based input stage 3302 of example SA assembly 3400 is coupled to summing nodes 3309A, 3309B, which feed into respective CMOS-based output gain legs 3402A, 3402B that each comprise a plurality of CMOS FETs. By way of illustration, output gain leg 3402A includes an NMOS device M1 coupled to a PMOS device M4 configured as a source follower, wherein two current sources I4 3404 and I5 3406 are respectively coupled to the drain terminals of M1 and M4. In similar fashion, output gain leg 3402B includes an NMOS device M2 coupled to a PMOS device M5 as a source follower, wherein two current sources I7 3408 and I6 3410 are respectively coupled to the drain terminals of M2 and M5. In one arrangement, a common NMOS device M3 may be coupled to the source terminals of M1 and M2 to form a respective differential amplifier for the corresponding output gain leg 3402A/3402B. Further, the summing nodes 3309A, 3309B are coupled to respective gate terminals of M1 and M2 devices. Accordingly, skilled artisans will recognize that power-optimized amplifier A1 of the embodiment shown in FIG. 33 may be implemented using M1 and M3 to form a first differential amplifier of the output gain leg 3402A, wherein M1 is loaded by I4 3404 and configured to drive a source follower, M4, that is biased by I5 3406. Likewise, power-optimized amplifier A2 of the embodiment shown in FIG. 33 may be implemented using M2 and M3 to form a second differential amplifier of the output gain leg 3402B, having I7 3408 to serve as a load to M2 and I6 3608 as a bias to M5 operating as a source follower. With respect to driving the output nodes of the SA assembly 3400, the drain terminals of PMOS devices M4 and M5 are respectively coupled to OUT_PLUS 3339A and OUT_MINUS 3339B, which may be coupled to an ADC circuit as previously noted.

Skilled artisans will further recognize upon reference hereto that whereas the output stage of the foregoing embodiment of SA 3400 is implemented using CMOS devices M1-M5, additional and/or alternative embodiments may include different circuit configurations, device combinations, and the like, including, for example, reversing the polarities of the FETs (i.e., replacing NMOS devices with PMOS devices and/or vice versa), device modes (e.g., enhancement mode vs. depletion mode), and the like. In a still further variation, an example SA assembly may include an arrangement where a common M3 between the differential amplifiers is not used (e.g., uncoupled differential amplifier implementation). However, certain combinations of amplifiers (e.g., involving true op amps) may be less attractive due to the trade-offs involving setting time vs. operating currents, for example.

Referring to the embodiments of FIG. 33 and FIG. 34 together, additional description relating to an example SA assembly's general operation, implementation, advantageous features, etc. will now be set forth immediately below.

In one arrangement, Q1 and Q2 devices of the input stage 3302 may comprise a matched pair of noise-optimized bipolar transistors, which may be configured to receive their operating current from source I1 3333. In one example implementation, Q1 and Q2 may comprise BJT devices each having a high forward transfer function, e.g., with a high forward gain factor (β) of 400 or more. By way of illustration, I1 may be in the range of about 30 μA to 80 μA although other current values may be implemented in some SA designs. In one arrangement, the voltage levels at the respective base terminals of Q1 and Q2 (i.e., VB), which are configured as input nodes INPUT_PLUS 3303A and INPUT_MINUS 3303B, may be (pre)set or (pre)established at a reference voltage (e.g., a configurable voltage level depending on the expected/desired performance characteristics of the SA assembly in a deployment scenario). For example, a V_{REF} value of about 1.0 V to 2.0 V may be provided in an illustrative embodiment, wherein an example differential input signal may represent a biosignal's peak-to-peak swing. Depending on implementation, the differential input signal, i.e., the difference in the voltage values (AV) between INPUT_PLUS 3303A and INPUT_MINUS 3303B, may range from 10 μV to 20 mV in a peak-to-peak measurement.

In one arrangement, the voltage difference (ΔV) between INPUT_PLUS and INPUT_MINUS nodes is operative to result in a difference in the respective collector currents, which is typically 9.5 μA per 10 mV of the voltage difference in an example implementation. For Q1, this collector current may be manifested at the output of A1 3304A, where A1, R1, and C1 may be operative as a first transimpedance amplifier with a gain of R1 volts per amp and a low-pass corner frequency of 1/(2π*R1*C1). In one arrangement utilizing R2=R1 and C2=C1, with respect to each respective output gain leg, the same performance behavior may be achieved in a second transimpedance amplifier comprising A2, R2, and C2. In an example implementation with a typical value of R1 being 127 kΩ and C1 being 470 pF, the gain of each leg may be determined as 0.127 V per μA. Given a current imbalance of 9.59 μA at 10 mV of input, the differential output voltage is around 1.21 V corresponding to a gain of 121 or thereabouts in an example implementation.

Because each/either of the first and second transconductance amplifiers may exhibit gain for the totality of the net current seen at its respective summing node 3309A/3309B, some current may be canceled in an example implementation via the addition of respective current sources, I2 3308A and I3 3308B. It should be appreciated that such an arrangement may be advantageously configured to remove a substantial offset voltage which would otherwise appear at the outputs, thereby allowing the amplifiers to have increased gain without their outputs saturating.

In one implementation, I2 3308A and I3 3308B may be varied via a configurable trimming operation (e.g., facilitated by or under the programmatic control of I/O driver circuitry) such that amplifiers A1 and A2 are configured to achieve nominal output voltages, which are typically around 2.0 V to 3.0 V when V_{DD} is around 3.0 V to 3.8 V. Further, because of the off-die trimming capability of I2 3308A and I3 3308B, the nominal output voltages of A1 and A2 may be configured to be approximately equal despite production variations in the CMOS ASIC chip and/or production variations in the BJT devices, e.g., V_{BE} variations of Q1 and Q2 as previously noted.

In some additional and/or alternative embodiments, additional trimming operations of current references may be required to ensure that the current consumption of the SA assembly is optimized for a particular deployment scenario. By way of example, operating current values of approximately 120 μA may be achieved, with roughly 50 μA passing through I1 in an implementation.

In one arrangement, the RC components, e.g., R1, R2, C1 and C2, may be provided as components external to the CMOS ASIC chip, as previously noted. It should be appreciated that such a configuration may be particularly advantageous in an example arrangement as this allows accurate gain and filter cut-off frequencies to be achieved without the silicon overhead and/or the circuitry needed for trimming and associated die area consumption on the same chip.

In the example embodiment depicted in FIG. 34, the transconductance amplifiers are implemented using MOSFET-based configurations although additional configurations based on other semiconductor technologies may also be achieved in some arrangements. Further, whereas example current sources are representatively depicted as ideal devices, it should be appreciated that the requisite current sources may be implemented or realized in several ways without undue experimentation. Skilled artisans will further recognize that contributions from the various components and circuit elements may need to be taken into consideration while designing an example SA assembly's equivalent input noise performance. In addition, where common resources are implemented in a circuit arrangement, e.g., common mode configuration, an implementation may introduce undesired feedback, which may need to be accounted for in a design. For instance, feedback between the elements I4 3404, I5 3406, I6 3410, and I7 3408 and the two trimmable elements, I2 3308A and I3 3308B may be difficult to control if they share resources and/or common paths depending on the particular example implementation.

In one example, M1 and its load, I4 3404, may be configured to form a high impedance/high gain amplifier (e.g., A1), which is buffered by M4 and its bias source I5 3406. In like fashion, A2 may be configured as a high impedance/gain amplifier based on M2 and its load I7 3408, buffered by M5 and its bias source I7 3410. In one arrangement, the sources of M1 and M2 may be configured to operate at an operating point established by M3 (e.g., in a coupled differential amplifier configuration) such that the operating point of the respective summing node 3309A, 3309B is approximately V_{REF}. In the example embodiment of FIG. 34, I12 3412 is configured to provide a supply current that is split between the FET devices forming the coupled differential amplifiers, i.e., M1, M2, and M3. Further, because M1 and M2 are operative to control respective summing nodes 3309A/3309B with equal and opposite input currents, the current through M3 is relatively constant in an example implementation.

In an alternative circuit arrangement, an additional transistor may be provided such that two separate and uncoupled differential amplifiers may be implemented, each with its own current source. However, such a configuration may impose the cost of additional bias current, which may add to further complexity. For example, it will be appreciated that when optimizing an example SA circuit design, the noise performance of M1 and M2 (and any additional devices) may be as critical as the performance of the devices comprising trimmable sources I2 3308A and I3 3308B in an example implementation.

In a further variation, suitable circuitry may be provided to modulate gain control of the SA assembly such that an input signal range of 1.0 μV to 20 mV may be accommodated for interfacing with a low-resolution ADC block that may be provided as part a suitable diagnostic/measurement circuitry. In an example arrangement, such circuitry for modulating or affecting gain control may be configured to vary the bias current of any long-tailed pair (LTP) stage within the SA assembly such that the transconductance of the LTP stage(s) is controlled, including, e.g., the BJT-based input stage being modulated in a preferential or prioritized manner.

Although not explicitly shown, it should be recognized that alternative circuit topologies may be utilized in an SA assembly to achieve either a single-ended or differential output representing the difference currents through Q1 and Q2. In addition, a single-ended input implementation utilizing only one BJT in the input stage may also be implemented for purposes of the present disclosure without undue experimentation.

FIG. 35 depicts an example biostimulation system including to an SA circuit of the present disclosure for coupling with a two-electrode arrangement and associated switching components according to an embodiment. As illustrated, biostimulation system 3500 includes a low-noise, low-power SA assembly 3502 having a differential input pair 3504A, 3505B and a differential output pair 3506A, 3506B, wherein the differential output pair 3506A, 3506B is operative to drive a differential input stage of an ADC as set forth hereinabove in detail. Each of the inputs 3504A, 3504B is coupled to a respective electrode (not specifically shown) by way of a conductive element 3508A, 3508B via respective capacitive elements C3 3510A and C4 3510B. Each conductive element 3508A, 3508B (hence the corresponding input nodes 3504A, 3504B) may be switchably coupled to a reference voltage (V_{REF}) via respective switch elements SW1 3512A, SW2 3512B. In one arrangement, C3 3510A and C4 3510B serve to shunt feed a differential signal collected by the electrodes, which may be selected via analog switches of a switch matrix of the IPG/IMD (e.g., switch matrix 3162 shown in the embodiment of FIG. 31 or switch matrix 3335 shown in the embodiment of FIG. 33). Resistive elements R3 3514A and R4 3514B may be utilized to supply bias current to the BJT input stage of SA assembly 3502, which typically requires a few tens of nanoamps in an example implementation. In one arrangement, SW1 3512A and SW2 3512B may comprise analog switches that may be utilized as a pair to rapidly charge and/or discharge C3 3510A and C4 3510B such that an operating point is rapidly established (e.g., within a predetermined/configurable time window), including in the situation where stimulation signals may have been recently applied to the selected electrodes. Depending on implementation, R3 3514A and R4 3514B may either be omitted or operated in a mode in which bias current to the BJTs is momentarily delivered via SW1 3512A and SW2 3512B (e.g., within a short duration) and maintained by capacitors C3 3510A and C4 3510B for a few milliseconds or tens of milliseconds, which may be activated together in an example embodiment.

In a further implementation, an additional switch SW3 3512C may be coupled between conductive elements 3508A, 3508B. Operationally, in one arrangement, SW1 3512A and SW2 3512B decrease the time associated with charging C3 3510A and C4 3510B upon applying power to the SA assembly 3502, while SW3 3512C serves to remove any accumulated charge between C3 3510A and C4 3510B subsequent to stimulation and prior to processing a biosignal, e.g., an ECAP signal. Should SW3 3512C fail to discharge the capacitors within the desired time, a compensating offset may be introduced into the biostimulation system 3500 to ensure that the output of the SA assembly 3502 is within the operating range of the ADC.

Skilled artisans will recognize that the foregoing RC arrangements associated with the respective input nodes 3504A/3504B of an example SA assembly 3502 are operable as high-pass filters configured to facilitate rapid charging/discharging operations that may be necessary where certain electrodes are configured to supply stimulation pulses in a stimulation event that may be followed by a sensing event wherein the previously energized electrodes are reconfigured to sense biosignals generated in response (e.g., ECAPs). In one example configuration, therefore, the base terminals of the BJTs may be respectively coupled to corresponding R-C junctions 3516A, 3516B of the RC high-pass filters (i.e., R3C3 filter and R4C4 filter) such that the time-varying potential of signal source(s) is capacitively coupled to the respective transistor(s) input. Furthermore, the other termini of the resistors R3 3514A and R4 3514B are terminated to a reference voltage (V_{REF}) such that the base terminal voltages (V_{BE}) of the BJT devices are driven to a steady state voltage approximating the reference voltage according to an example implementation. Also, resistors R3 3514A and R4 3514B of the RC high-pass filters are disposed in parallel to the corresponding analog switches SW1 3512A and SW2 3512B such that the actuation of the switching elements is operative to reduce the time constant(s) of the RC high-pass filters in order to facilitate the rapid charging/discharging operations associated with the selected electrodes of the biostimulation system 3500.

Skilled artisans will recognize upon reference hereto that example SA embodiments advantageously utilize noise-optimized bipolar transistors as active elements within an LTP arrangement such that one or more design criteria may be met. First, the electrical noise introduced by the LTP stage is sufficiently low as to assure that the accumulation of noise generated by all circuit elements represents less than a predetermined root mean square (RMS) value of equivalent input noise, e.g., 1.0 μV RMS of equivalent input noise in some embodiments. Second, the gain associated with the LTP stage is sufficient to ensure that the noise produced by the CMOS circuit elements does not represent more than some small, predetermined amount of the total equivalent noise (e.g., less than 25% of the cumulative equivalent input noise). Third, the LTP stage is operable with voltages and currents commensurate with a power budget supported by a battery-operated IPG, such as, e.g., 3.4 V and 50 μA amps, respectively, in an example implementation. Additionally, the physical area of the LTP stage, exclusive of the CMOS elements of the output gain stage, is sufficiently compact, e.g., 8 mm² or thereabouts, in order to ensure compatibility with the dimensions and other form factor constraints of a state-of-the-art IPG/IMD/NIMI device. It should be appreciated that the foregoing list of criteria is not an exhaustive list and, depending on implementation, more or fewer criteria and/or different sets of criteria even, may be contemplated in designing an SA assembly in accordance with the teachings herein.

In another aspect of the present disclosure, embodiments of an SA assembly may be configured to provide a means to compensate for production/performance variation in the various stages (e.g., the BJTs' base-emitter voltages, the CMOS FET stages, etc.) as previously noted. In some additional/alternative example embodiments, suitable compensation may be is achieved by enabling or disabling additional MOS transistors in the load of the bipolar LTP stage consistent with the method of U.S. Pat. No. 4,987,327 A, or alternately, via utilization of MOS transistors in place of Zener fuses as proposed in U.S. Pat. No. 4,717,888 A, each of which is hereby incorporated by reference herein.

Based on the foregoing, it will be appreciated that IPG 12/3150 or another suitable implantable pulse generator may be advantageously adapted to include SA circuitry for amplifying neural or other biosignal activity via suitable hybridization of semiconductor technologies such that an IPG/IMD may be realized that is compact, produces low equivalent input noise, and consumes currents commensurate with a battery/power supply of the implantable device. As set forth in detail above, example hybridization technologies for providing a synergistic combination may preferably involve low noise bipolar transistor(s) coupled with a low-power consumption CMOS ASIC, wherein a compact circuitry arrangement using a hybrid design may advantageously result in low power consumption (typically about 120 μA as an example) while exhibiting an equivalent low input noise of about 0.65 μV over a 1.0 Hz to 3.0 kHz output bandwidth as an example when driven from a 3 kΩ source.

In some embodiments, a hybrid circuit design for amplifying neural or other low amplitude biosignals may be advantageously configured to improve equivalent input noise performance by a factor of three times (3X) that of circuits based upon state-of-the-art operational amplifiers utilizing similar current. Likewise, such a circuit design may exceed the noise performance of instrumentation amplifiers by a factor of six (6X) according to some example embodiments of the present disclosure.

In some embodiments, a hybrid amplifier in an IPG for sensing biosignals (preferably neural biosignals) may comprise noise-optimized bipolar transistor(s) fabricated on one die and a power-optimized CMOS IC on another die, whereby different levels of trimming control may be advantageously provided, which facilitates a wide range of application scenarios as noted previously. Still further, an example hybrid amplifier arrangement may be configured to provide an improved trade-off between equivalent input noise and supply current.

Although the foregoing hybrid SA circuitry arrangements and related embodiments have been described in detail for integration with an implantable pulse generator, sensing circuitry according to any of the embodiments discussed herein may be used for any suitable implantable or semi-implantable medical device as noted previously. For example, cardiac sensing devices, cardiac rhythm management (CRM) devices, implantable defibrillators, glucose monitoring devices, insulin infusion devices, and/or the like may employ sensing circuitry as discussed herein. Additionally, an embodiment of hybrid SA assembly of the present disclosure may be deployed in connection with various therapies including, e.g., SCS therapy, a dorsal root ganglion (DRG) stimulation therapy, a deep brain stimulation (DBS) therapy, a cochlear stimulation therapy, a cardiac pacemaker therapy, a cardioverter-defibrillator therapy, a cardiac rhythm management (CRM) therapy, an electrophysiology (EP) mapping and radio frequency (RF) ablation therapy, an electroconvulsive therapy (ECT), a repetitive transcranial magnetic stimulation (rTMS) therapy, and a vagal nerve stimulation (VNS) therapy, etc. Also, any suitable biosignal may be processed using sensing circuitry described herein such as ECAP signals, local field potential signals, EMG signals, EEG signals, cardiac signals, respiratory-related signals, and/or the like.

Further, although the input and output stages of a hybrid SA assembly have been described in reference to BJTs and CMOS FETs, other semiconductor technologies may be used in additional and/or alternative embodiments, e.g., involving BiCMOS, linear BiCMOS (LBC), etc., which may allow integration of noise and power optimization in a single IC for purposes of at least some embodiments herein.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate based on the the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

In the above description of various embodiments of the present disclosure, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and may not be interpreted in an idealized or overly formal sense expressly so defined herein.

At least some example embodiments are described herein with reference to one or more circuit diagrams/schematics, block diagrams and/or flowchart illustrations. It is understood that such diagrams and/or flowchart illustrations, and combinations of blocks in the block diagrams and/or flowchart illustrations, can be implemented by any appropriate circuitry configured to achieve the desired functionalities. Accordingly, example embodiments of the present disclosure may be embodied in hardware and/or in software (including firmware, resident software, micro-code, etc.) operating in conjunction with suitable processing units or microcontrollers, which may collectively be referred to as “circuitry,” “a module” or variants thereof. An example processing unit or a module may include, by way of illustration, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Array (FPGA) circuits, any other type of integrated circuit (IC), and/or a state machine, as well as programmable system devices (PSDs) employing system-on-chip (SoC) architectures that combine memory functions with programmable logic on a chip that is designed to work with a standard microcontroller. Example memory modules or storage circuitry may include volatile and/or non-volatile memories such as, e.g., random access memory (RAM), electrically erasable/programmable read-only memories (EEPROMs) or UV-EPROMS, one-time programmable (OTP) memories, Flash memories, static RAM (SRAM), etc., including other types of persistent memory.

Further, in at least some additional or alternative implementations, the functions/acts described in the blocks may occur out of the order shown in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Furthermore, although some of the diagrams include arrows on communication paths to show a primary direction of communication, it is to be understood that communication may occur in the opposite direction relative to the depicted arrows. Finally, other blocks may be added/inserted between the blocks that are illustrated.

It should therefore be clearly understood that the order or sequence of the acts, steps, functions, components or blocks illustrated in any of the flowcharts depicted in the drawing Figures of the present disclosure may be modified, altered, replaced, customized or otherwise rearranged within a particular flowchart, including deletion or omission of a particular act, step, function, component or block. Moreover, the acts, steps, functions, components or blocks illustrated in a particular flowchart may be inter-mixed or otherwise inter-arranged or rearranged with the acts, steps, functions, components or blocks illustrated in another flowchart in order to effectuate additional variations, modifications and configurations with respect to one or more processes for purposes of practicing the teachings of the present patent disclosure.

Although various embodiments have been shown and described in detail, the claims are not limited to any particular embodiment or example. None of the above Detailed Description should be read as implying that any particular component, element, step, act, or function is essential such that it must be included in the scope of the claims. Where the phrases such as “at least one of A and B” or phrases of similar import (e.g., “A and/or B”) are recited or described, such a phrase should be understood to mean “only A, only B, or both A and B.” Reference to an element in the singular is not intended to mean “one and only one” unless explicitly so stated, but rather “one or more.” Moreover, the terms “first,” “second,” and “third,” etc. employed in reference to elements or features are used merely as labels, and are not intended to impose numerical requirements, sequential ordering or relative degree of significance or importance on their objects. All structural and functional equivalents to the elements of the above-described embodiments that are known to those of ordinary skill in the art are expressly incorporated herein by reference and are intended to be encompassed by the present claims. Accordingly, those skilled in the art will recognize that the exemplary embodiments described herein can be practiced with various modifications and alterations within the spirit and scope of the claims appended below.

1. An implantable pulse generator (IPG) for generating electrical pulses to stimulate a neural tissue of a patient, comprising:
one or more battery components for powering the IPG;
pulse generating circuitry for generating electrical pulses;
a header structure with a plurality of electrical connections adapted to contact one or more terminals of one or more stimulation leads or one or more lead extensions coupled to respective ones of the one or more stimulation leads, wherein the one or more stimulation leads each include a plurality of electrodes; and
sensing circuitry for sensing one or more biosignals associated with the neural tissue, wherein the sensing circuitry comprises an amplifier for amplifying the one or more biosignals, the amplifier including a noise-optimized input stage coupled to a power-optimized output stage.

2. The IPG as recited in claim 1, wherein the noise-optimized input stage comprises one or more bipolar junction transistors (BJTs) formed on a first semiconductor die and the power-optimized output stage comprises one or more complementary metal-oxide-semiconductor (CMOS) field effect transistors (FETs) formed on a second semiconductor die.

3. The IPG as recited in claim 2, wherein the one or more BJTs comprise BJTs having a forward gain greater than a predetermined value.

4. The IPG as recited in claim 2, wherein the one or more CMOS FETs are configured to operate as a bias current source for providing a bias current to the input stage.

5. The IPG as recited in claim 4, wherein the bias current source is configured as a variable current source operable to control a gain associated with the input stage.

6. The IPG as recited in claim 2, wherein the one or more CMOS FETs are configured to operate as a load with respect to the input stage.

7. The IPG as recited in claim 6, wherein the load comprises a variable load operable to compensate for production variation in the one or more BJTs forming the input stage or in the one or more CMOS FETs forming the output stage of the amplifier.

8. The IPG as recited in claim 2, wherein the one or more CMOS FETs are configured to amplify one or more signals generated by the input stage responsive to the one or more biosignals driving the input stage.

9. The IPG as recited in claim 1, wherein the one or more biosignals comprise at least one evoked compound action potential (ECAP) signal generated by the nerve tissue in response to receiving one or more stimulation pulses.

10. The IPG as recited in claim 1, wherein the one or more biosignals comprise at least local field potential signal associated with the nerve tissue.

11. The IPG as recited in claim 1, wherein the pulse generating circuitry is operable to be configured responsive to a control signal generated by a diagnostic circuit driven by the one or more biosignals sensed by the sensing circuitry.

12. The IPG as recited in claim 1, further comprising switching circuitry for selectively outputting generated stimulation pulses to one or more electrical connections and for selectively connecting one or more electrical connections to the sensing circuitry to sense the one or more biosignals using one or more electrodes of the one or more stimulation leads.

13. The IPG as recited in claim 1, wherein the power-optimized output stage is configured to drive a pair of differential output nodes coupled to an analog-to-digital converter (ADC) having a differential input.

14. The IPG as recited in claim 1, wherein the noise-optimized input stage is configured as a differential input stage operable to connect to a select pair of electrodes of the one or more stimulation leads.

15. The IPG as recited in claim 14, wherein the differential input stage includes a first input node and a second input node, the first input node coupled to a first high pass filter disposed between the first input node and a corresponding first electrode, the second input node coupled to a second high pass filter disposed between the second input node and a corresponding second electrode, the first and second high pass filters driven by a reference voltage.

16. The IPG as recited in claim 15, wherein the first and second high pass filters are each operated by a respective switch configured to facilitate charging or discharging of a corresponding capacitive element of the first and second high pass filters within a predetermined time window.

17. The IPG as recited in claim 1, wherein the power-optimized output stage is configured for operating the amplifier with a current less than around 100 μA to 150 μA.

18. The IPG as recited in claim 1, wherein the noise-optimized input stage is configured for operating the amplifier with a root mean square (RMS) equivalent input noise level of less than around 1.0 μV RMS to 1.5 μV RMS.

19. An implantable medical device (IMD) for sensing biosignals of a patient, the IMD comprising:
a power supply for powering the IMD;
one or more sensing elements; and
sensing circuitry including an amplifier comprising a first circuit portion formed of one or more bipolar junction transistors (BJTs) disposed on a first semiconductor die and a second circuit portion electrically coupled to the first circuit portion, the second circuit portion formed of a plurality of complementary metal-oxide-semiconductor (CMOS) field effect transistors (FETs) disposed on a second semiconductor die, the first circuit portion configured to receive a biosignal from the patient via one or more sensing elements.

20. The IMD as recited in claim 19, wherein the one or more BJTs each comprise a low noise BJT having a forward current gain value greater than 400.

21. The IMD as recited in claim 19, wherein one or more of the CMOS FETs are configured to operate as a bias current source for providing a variable bias current to the first circuit portion.

22. The IMD as recited in claim 19, wherein one or more of the CMOS FETs are configured to operate as a load with respect to the first circuit portion.

23. The IMD as recited in claim 19, wherein the first circuit portion is configured as a differential input stage operable to connect to a select pair of sensing elements.

24. The IMD as recited in claim 23, wherein the second circuit portion is configured as an output stage operable to drive a pair of differential output nodes coupled to an analog-to-digital converter (ADC) having a differential input.

25. The IMD as recited in claim 24, wherein the second circuit portion comprises a first amplifier configured to operate in association with a first RC circuit as a first transimpedance amplifier and a second amplifier configured to operate in association with a second RC circuit as a second transimpedance amplifier, the first and second amplifiers driving the pair of differential output nodes.

26. The IMD as recited in claim 25, wherein the first and second RC circuits are configured operate with matching corner frequencies.

27. The IMD as recited in claim 25, wherein the first and second RC circuits are disposed as trimming circuitry external to the second semiconductor die.

28. The IMD as recited in claim 19, wherein at least a subset of the CMOS FETs are configured into a pair of coupled differential amplifiers having a common current source.

29. The IMD as recited in claim 19, wherein at least a subset of the CMOS FETs are configured into a pair of uncoupled differential amplifiers, each having a corresponding current source.
